(19) United States
(12) Patent Application Publication
(10) Pub. No.: US 2026/0294545 A1
(43) Pub. Date: Oct. 1, 2026

(54) METHOD AND APPARATUS FOR A SIMULATED WEIGHT-BEARING POSITIONING SYSTEM

(71) Applicant: Orthios Innovations, LLC, Kearny, NJ (US)

(72) Inventors: Richard Adelson, North Bergen, NJ (US); Daniel Cheney, Downingtown, PA (US); David Pancratz, Helotes, TX (US); Keith Peeples, San Antonio, TX (US)

(73) Assignee: Orthios Innovations, LLC, Kearny, NJ (US)

(21) Appl. No.: 19/700,933

(22) Filed: Jun. 8, 2026

Related U.S. Application Data
(63) Continuation-in-part of application No. 18/605,131, filed on Mar. 14, 2024.
(60) Provisional application No. 63/497,709, filed on Apr. 22, 2023.
(60) Provisional application No. 63/588,331, filed on Oct. 6, 2023.
(60) Provisional application No. 63/824,547, filed on Jun. 16, 2025.
(60) Provisional application No. 63/902,861, filed on Oct. 21, 2025.

(51) Int. Cl. A61B 34/10; A61B 6/04

(52) U.S. Cl. CPC A61B 34/10; A61B 6/0421; A61B 6/0492; A61B 2034/104

(57) ABSTRACT

A simulated weight-bearing positioning apparatus includes a base configured to support at least a portion of a patient's leg and a footpad movable relative to the base between a stowed position and a deployed position. The footpad includes a patient-contacting surface configured to contact a plantar surface of the patient's foot. One or more guide features guide movement of the footpad relative to the base. The footpad may translate relative to the base, rotate relative to the base, and engage the base at a selected deployed angle. A locking feature retains the footpad in the deployed position so that the patient's foot may be maintained in a simulated weight-bearing position for imaging, surgery, or another clinical procedure. In some embodiments, the apparatus permits repeated deployment and re-stowing of the footpad during a procedure.

### BACKGROUND OF THE INVENTION

### 1. Field of the Invention

The present invention relates to an apparatus capable of creating an effective and reproducible simulated weight-bearing position for the foot and ankle of a sedated patient, an awake but non-ambulatory patient, or an ambulatory but resting patient. The invention also includes a method of use for creating this simulated weight-bearing position.

### 2. Description of the Related Art

During foot and ankle surgery, or in a clinical setting for non-ambulatory or resting patients, orthopedic surgeons frequently desire to see the normal positioning of bones and joints of a patient's foot. A patient resting or sedated on a bed does not have their foot in a normal, weight-bearing position. Instead, the foot of a resting or sedated patient would have the toes extended downward and outward, with bones oriented differently than while walking. In this position, the surgeon cannot visualize the effectiveness of surgery or walking anatomy. To view a patient's foot in a normal position, the physician desires to create a simulated weight-bearing position for the foot, and then hold the foot in this position while performing imaging studies. Imaging can include x-rays (digital such as C-arm or film-based), CT scans, or MRI images. The physician would like this simulated weight-bearing position to be reproducible, so that the images have the same orientation and relative bone positioning as the surgery progresses. In addition, as the patient heals post-operatively or in a clinical setting, the physician can continue to create a simulated weight-bearing position with the same orientation and relative positioning. Full view simulated weight-bearing images assist the surgeon to assure that any fixation that is being placed in the foot is in the proper position in the foot's normal position. In addition, simulated weight-bearing images provide an accurate assessment of the surgical correction that took place, and will be predictive of the series of post-operative x-rays that will be taken as the patient heals.

Existing systems do not adequately provide an effective and reproducible simulated weight-bearing position for a patient's foot while allowing hands-free imaging. There are also no adequate systems available that allow the patient's foot to be positioned in multiple planes. Existing solutions involve manually using a metallic or plastic object to press against the patient's foot, with different amounts of force and from different angles, creating a non-reproducible and unpredictable outcome. In addition, existing solutions involve operating room or healthcare providers to be in the field of the imaging equipment, creating unnecessary radiation or electromagnetic field exposure. There are also no systems available to hold and position a patient's foot in a desired position, with or without a load. There are also no systems available that allow surgical instruments to be mounted so that surgery can be conducted in a weight-bearing position.

Accordingly, a system is described herein for creating reproducible, repeatable simulated weight-bearing of a foot in a resting or sedated patient, while allowing healthcare staff to remain clear of the imaging field of view. Various configurations of the apparatus and methods of use for the system are also presented.

### SUMMARY OF THE INVENTION

The present invention relates to a simulated weight-bearing positioning apparatus and related methods for positioning a patient's foot in a reproducible simulated weight-bearing position for imaging, surgery, or other clinical procedures. The apparatus includes a base configured to support at least a portion of a patient's leg and a footpad movable relative to the base between a stowed position and one or more deployed positions. The footpad is deployed through a controlled sequence of movement. The footpad may translate relative to the base, rotate relative to the base, and then engage the base at a selected angle. Guide features including rails, channels, pins, slots, stops, or other cooperating features may control the motion and permit the footpad to be repeatedly deployed and re-stowed during a procedure. The footpad is stabilized in a deployed position by spaced-apart engagement between the footpad and the base. For example, an upper engagement feature may engage a locking slot while a lower engagement feature engages a lower receiving feature. Forces applied by the patient's foot may be resisted by these spaced-apart engagement features, thereby maintaining the footpad at the selected angle.

In some embodiments, the apparatus includes a profiled lower pin and a corresponding slot arrangement. The profiled lower pin may prevent downward movement of the footpad in one rotational orientation and permit downward movement after the footpad has been rotated to another orientation. This arrangement can provide a controlled kinematic sequence for moving the footpad from a stowed position to a locked deployed position.

In some embodiments, the invention also includes, or may be used with, one or more wedges configured to incline or elevate the apparatus; radiopaque marker strips configured to provide imaging reference lines; access openings or slots in the footpad; and attachment features configured to receive straps, slides, stabilizers, instrument guides, or surgical instruments.

In some embodiments, the footpad may include an articulating structure having upper and lower footpad portions connected by a pivot. The articulating structure may permit the patient's foot to be positioned or loaded in more than one plane.

A method of use includes placing the apparatus beneath at least a portion of a patient's leg, moving the footpad from a stowed position to a selected deployed position, positioning the patient's foot against the footpad, and maintaining the foot in a simulated weight-bearing position while imaging or surgery is performed. The footpad may be repeatedly stowed and redeployed during the procedure.

### BRIEF DESCRIPTION OF THE DRAWINGS

FIG. 1 shows a front isometric view of a simulated weightbearing apparatus in position for patient use.

FIG. 2 shows a right side view of a simulated weightbearing apparatus in position for patient use.

FIG. 3 shows a right side view of a simulated weightbearing apparatus in position for patient use, with a representative patient foot and leg.

FIG. 4 shows a top view of a simulated weightbearing apparatus in position for patient use.

FIG. 5 shows a back view of a simulated weightbearing apparatus in position for patient use.

FIG. 6 shows a bottom view of a simulated weightbearing apparatus in position for patient use.

FIG. 7 shows a front view of a simulated weightbearing apparatus in position for patient use.

FIG. 8 shows an isometric rear view of a simulated weightbearing apparatus in position for patient use.

FIG. 9 shows a front isometric view of a base.

FIG. 10 shows a top isometric view of a base.

FIG. 11 shows a front isometric view of a footpad.

FIG. 12 shows an isometric view of a simulated weightbearing apparatus in position for patient use, with attachment mounts in place.

FIG. 13 shows a back isometric view of a simulated weightbearing apparatus in position for patient use, with attachment mounts in place.

FIG. 14 shows an isometric view of instrument guides and various instruments.

FIG. 15 shows a front isometric view of a simulated weightbearing apparatus in position for patient use, with attachment mounts and instrument guides in place.

FIG. 16 shows a top isometric view of a simulated weightbearing apparatus in position for patient use, with attachment mounts and instrument guides in place.

FIG. 17 shows a front isometric view of a simulated weightbearing apparatus in position for patient use, with attachment mounts. instrument guides, and instruments in place.

FIG. 18 shows a top view of a footpad with representative patient foot and toes, and slides holding foot in place.

FIG. 19 shows an isometric view of a latch.

FIG. 20 shows a close-up rear isometric view of a base with base slot visible.

FIG. 21 shows a close-up rear isometric view of a base with latch slot visible but latch removed for clarity.

FIG. 22 shows a close-up rear isometric view of a base with latch and latch slot.

FIG. 23 shows a right side view of a base with location of cross-section A-A identified.

FIG. 24 shows a rear view of a base at cross-section A-A.

FIG. 25 shows a close-up right side isometric view of a footpad and base in position for patient use.

FIG. 26 shows a close-up top right isometric view of a footpad and base in position for patient use.

FIG. 27 shows a front right isometric view of a simulated weightbearing apparatus in position for patient use.

FIG. 28 shows a front right isometric view of a simulated weightbearing apparatus in position for patient use.

FIG. 29 shows a front right isometric view of a simulated weightbearing apparatus in angled position for patient use.

FIG. 30 shows a top view of a simulated weightbearing apparatus in angled position for patient use.

FIG. 31 shows a close-up top view of the right heel cup 908 and surrounding area of a simulated weightbearing apparatus in angled position for patient use.

FIG. 32 shows a back view of a simulated weightbearing apparatus in angled position for patient use.

FIG. 33 shows a right side view of a simulated weightbearing apparatus in angled position for patient use.

FIG. 34 shows a bottom view of a simulated weightbearing apparatus in angled position for patient use.

FIG. 35 shows a front view of a simulated weightbearing apparatus in angled position for patient use.

FIG. 36 shows a right front isometric view of a simulated weightbearing apparatus in upright position for patient use.

FIG. 37 shows a close-up of details from FIG. 36.

FIG. 38 shows an isometric view of a footpad.

FIG. 39 shows a left isometric view of a wedge.

FIG. 40 shows a top view of a wedge.

FIG. 41 shows a bottom view of a wedge.

FIG. 42 shows a left side view of a wedge.

FIG. 43 shows a left side view of two wedges in an angled position.

FIG. 44 shows a right isometric view of two wedges in an angled position.

FIG. 45 shows a right isometric view of two wedges in angled position with a simulated weightbearing apparatus.

FIG. 46 shows a right side view of two wedges in angled position with a simulated weightbearing apparatus.

FIG. 47 shows a right isometric view of two wedges in an elevating position with a simulated weightbearing apparatus.

FIG. 48 shows a right side view of two wedges in an elevating position with a simulated weightbearing apparatus.

FIG. 49 shows a right isometric view of two wedges in an elevating position with a simulated weightbearing apparatus in position for patient use.

FIG. 50 shows a front view of two wedges in an elevating position with a simulated weight bearing apparatus with footpad in upright position.

FIG. 51 shows an isometric view of the lower base.

FIG. 52 shows an isometric view of a calf plate.

FIG. 53 shows an isometric bottom view of a calf plate.

FIG. 54 shows an exploded side view of a lower base, a footpad, and a calf plate.

FIG. 55 shows an exploded isometric view of a lower base, a footpad, and a calf plate.

FIG. 56 shows an assembled isometric view of a lower base, a footpad, and a calf plate.

FIG. 57 shows an isometric view of a third embodiment of a footpad.

FIG. 58 shows a side view of a third embodiment of a footpad.

FIG. 59 shows a close-up side view of a third embodiment of a footpad.

FIG. 60 shows a back view of a third embodiment of a footpad.

FIG. 61 shows a top view of a third embodiment of a footpad.

FIG. 62 shows an isometric view of a calf plate.

FIG. 63 shows an isometric view of a lower base.

FIG. 64 shows a right side view of a lower base.

FIG. 65 shows a top view of a lower base.

FIG. 66 shows an end view of a lower base.

FIG. 67 shows an isometric view of an assembled base without the footpad.

FIG. 68 shows an isometric view of a simulated weightbearing apparatus in the stowed position.

FIG. 69 shows an isometric view of a simulated weightbearing apparatus with footpad partially deployed.

FIG. 70 shows an isometric view from underneath of a simulated weightbearing apparatus with footpad partially deployed.

FIGS. 71A and 71B show isometric views including a close-up of a simulated weightbearing apparatus with footpad partially deployed.

FIGS. 72A and 72B show isometric views including a close-up of a simulated weightbearing apparatus with footpad partially deployed and rotated.

FIGS. 73A and 73B show isometric views including a close-up of a simulated weightbearing apparatus with footpad in the reclined position.

FIG. 74 shows an isometric view of a simulated weightbearing apparatus with footpad in the reclined position.

FIG. 75 shows an isometric view of a simulated weightbearing apparatus with footpad in the upright position.

FIG. 76 shows an isometric view with patient's leg and foot, of a simulated weightbearing apparatus with footpad in the upright position.

### DETAILED DESCRIPTION OF THE PREFERRED EMBODIMENTS

### Overview

### Purpose of the System

Some purposes of the apparatus and method in this system are as follows:
To allow a physician or technician to effectively orient and maintain the foot of a patient in a simulated weight-bearing position and permit surgery or imaging in that position.
To allow a physician or technician to orient the foot of a patient in multiple planes, or degrees of freedom, in a simulated weight-bearing position and permit surgery or imaging in that position.
To allow this positioning to occur quickly, and reproducibly, such that the imaging studies show the foot in the same position each time.
To allow images to be taken throughout the diagnostic, procedural, and follow up setting in order to improve the accuracy of the information that physicians are currently using in foot and ankle and other orthopedics specialties.
To allow placement of a patient's foot in a simulated weight-bearing position, and maintained in that position, in a hands-free manner so that operating room or staff are not exposed to imaging energy.
To allow the physician to position the foot at different angles and in different planes, and simulate weight-bearing.
To provide a system for mounting and positioning surgical instruments that can be used while the patient is in simulated weight-bearing.
To provide a system that allows sliders and stabilizers that orient and press the foot into the correct position.
To provide a system that allows the physician or technician to increase or decrease the angle of the patient's foot and leg, or elevate the foot and leg, for surgery or imaging.

Description of problem(s) that this System solves:
Weight-bearing imaging is important to study the functional position of the foot for proper evaluation of the patient's anatomy.
During foot surgery, the patient is unable to stand for imaging that would show true weight-bearing.
Physicians simulate weight-bearing by pressing against the foot of the prone patient, with their hands or any available flat surface. This method is imprecise and not easily repeated. An imaging study may have differences in angles and loading.
There are currently no standards of care for consistent simulated weight-bearing throughout orthopedics, or for non-ambulatory patients in any setting including the operating room, diagnostic imaging centers or physician offices. This system would allow for standards to be developed.
Healthcare professionals can use this system “hands-free”. Once set up, this system will provide the pressure necessary to simulate weight-bearing hands-free (no active interaction) allowing the healthcare professionals to remove themselves from exposure.
This system creates reproducible positioning and orientation of a patient's foot. Images taken at different time points will show the same orientation and relative positioning of bones and joints. Surgery performed will be more reproducible and predictable from the accurate positioning.
The system allows the physician to perform surgery while the patient's foot is in a weight-bearing and loaded position. In this way, bones are correctly oriented during the surgery.
The system provides movable or stationary attachment locations for numerous surgical instruments.
The system provides sliders that can orient the foot in a desired position.
The system provides wedges that can elevate the foot or change the angle of the foot.

Description on how the System is an improvement over existing technology:
Current methods do not create reproducible simulated weight-bearing positions. Current methods are manually applied, from different angles and with different amounts of pressure. Current methods are not reproducible if multiple x-rays are desired, at different stages during surgery and post-operatively.
Current methods require ambulatory patients that can get simulated weight-bearing xrays by standing on a specially designed platform.
The current methods used to simulate weight-bearing in most non-ambulatory settings require a healthcare professional to actively engage with the patient in order to hold their leg and foot in a proper position while the image is being taken thus exposing them to radiation.
The current methods used to simulate weight-bearing frequently involve radiopaque and/or metal components, reducing the quality and accuracy of the image.
Current methods do not provide a way to perform surgery with the foot in a weight-bearing position. Current methods also do not provide a way to perform surgery with the foot being loaded with a force.
Current methods do not allow the foot to be oriented and held in a desired position.

Some features of the System:
Fully radiolucent, except radiopaque marker strips allow for more accurate positioning and perspective on lateral radiograph images
Hands-free (once set in place)
Reproducible imaging with a flat surface pressing against the foot
Quick set and release of the footpad
Multi-use (autoclavable for operating room use, or non-sterile use)
Operates in different clinical settings: intraoperative, post-op, clinic
Folds flat to a working surface
Portable, with a handle
Allows entire foot visualization
Eliminates the need to continuously manipulate the patient's leg and foot intraoperatively
Patient comfort features such as rounded edges, cushions, etc.
Footpad able to adjust to various angles
Can be used for A/P or lateral imaging with no reassembly
Footpad features include heel support, guiderails for foot, texture, and holes for cleaning
Slots to receive belts for the table, leg, or foot, to stabilize the system
Slots to receive mounting jigs that can attach to surgical instruments
Sliders to press against the foot and orient it in a desired position
Wedges raise the foot or change the angle of the foot and leg
A separate kit of instruments or padding that can be used with the apparatus
A latch that slides up and down to hold the footpad in a stowed position
Articulations in the footpad allow for orientation of the patient's foot in multiple planes

In the description below and to avoid confusion, the first embodiment pertains to base 100, footpad 200, and calf plate 300. An articulating footpad embodiment includes footpad 250 with upper footpad 252, lower footpad 251, and pivot 253. A second embodiment pertains to base 900 with calf plate 901, lower base 902, and footpad 275. A third embodiment pertains to base assembly 1900, calf plate 1901, lower base 1902, and footpad 1275. In some embodiments, one or more radiopaque marker strips 230 are positioned on or within footpad 200, 275, or 1275. The marker strips 230 may provide a visible reference line during lateral imaging, may simulate a ground plane, and may assist the user in determining whether the image is properly aligned.

As used herein, stowed position 5 refers to a position in which the footpad is substantially stowed within the base for storage, sterilization, transport, or temporary clearance during a procedure. Upright position 10 refers to a deployed position in which the footpad is generally perpendicular to the calf plate. Angled position 13 refers to a deployed position in which the footpad is inclined relative to the calf plate, which can be greater or less than 90 degrees. Positions 15 and 18 refer to articulated configurations of footpad 250. Unless otherwise stated, the terms “base,” “base assembly,” “calf plate,” “lower base,” “footpad,” “guide feature,” “locking feature,” “engagement feature,” “receiving feature,” “slot,” “rail,” “channel,” “pin,” “stop,” and similar terms are used broadly. These structures may be formed integrally, attached separately, reversed between components, duplicated on opposite sides, or replaced with mechanically equivalent structures that guide, support, retain, position, or stabilize the footpad relative to the base. As used herein, the term “apparatus” may refer to the simulated weight-bearing device itself, including a base and a movable footpad. The term “system” may refer to the apparatus alone or to the apparatus in combination with one or more related components, accessories, or instruments, including wedges, straps, slides, stabilizers, instrument guides, surgical instruments, padding, radiopaque marker strips, or packaging. Unless otherwise stated, features described with respect to one apparatus, system, or embodiment may be used with any other apparatus, system, or embodiment described herein.

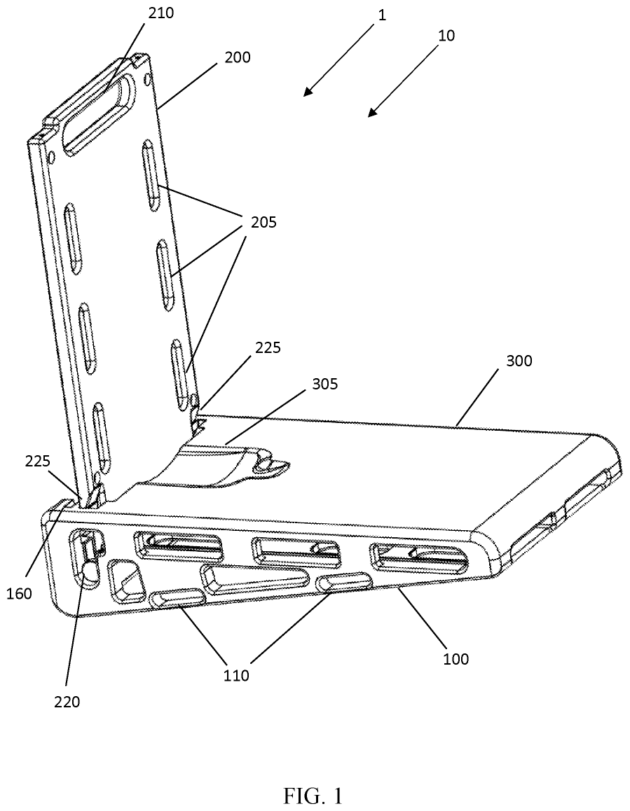

FIG. 1 displays a first embodiment of the simulated weightbearing apparatus 1. Major components include base 100, footpad 200 which is shown in upright position 10, and calf plate 300 which is the top surface of base 100. Strap slots 110 are through openings in base 100 to allow the base 100 to be strapped to the operating table, or to allow the patient's foot and leg to be strapped to the simulated weightbearing apparatus 1, or both. Leg strap slots 112 are similar through openings that can be used to strap the patient's foot and leg to the simulated weightbearing apparatus 1. The footpad 200 has a patient-contacting surface that contacts the bottom, sole, or plantar surface of the patient's foot and can apply a load to the foot.

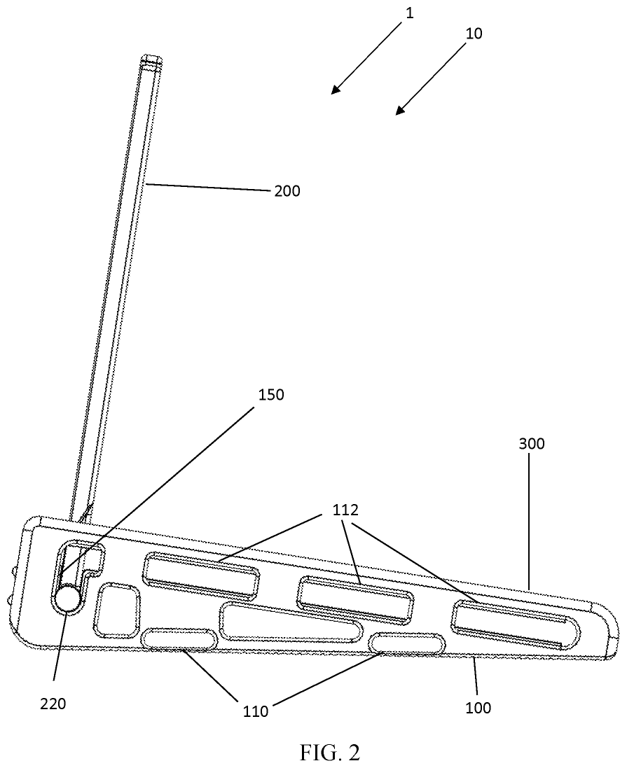

FIG. 2 displays a side view of apparatus 1. Calf plate 300 is the upper surface of base 100 and is a leg-supporting surface, in that it is designed to support a patient's leg and foot. Footpad 200 exerts a force on the patient's foot when it is in the upright position 10. Similar patient-contacting surfaces and leg-supporting surfaces may be provided in the other embodiments described herein, including footpad 250, footpad 275, footpad 1275, calf plate 901, and calf plate 1901.

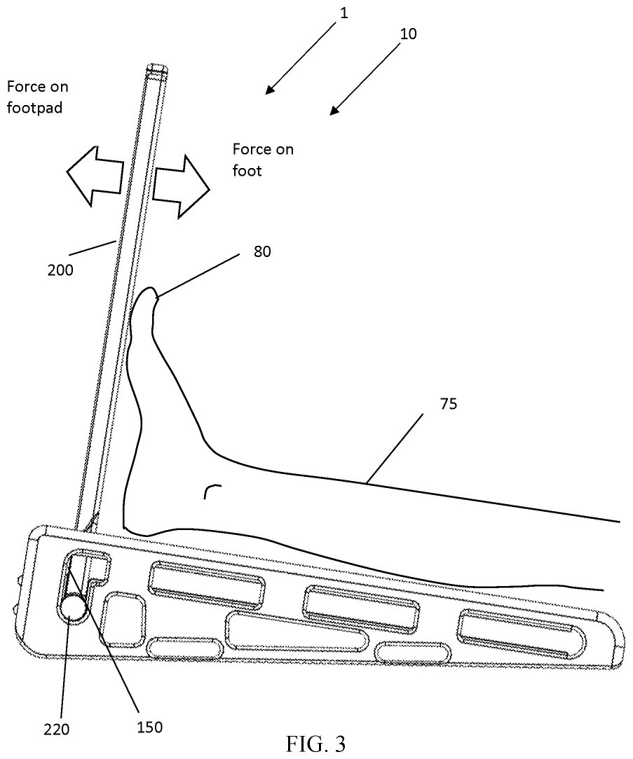

FIG. 3 shows a side view of apparatus 1 with typical patient leg 75 and patient foot 80 on calf plate 300 of base 100. When a physician or technician has positioned the foot 80 in the proper position, then footpad 200 in upright position 10 exerts a force on the bottom of foot 80. An equal and opposite force is also transmitted to footpad 200. A couple moment created by lower pin 220 and positioning edge 225 resists the force of foot 80.

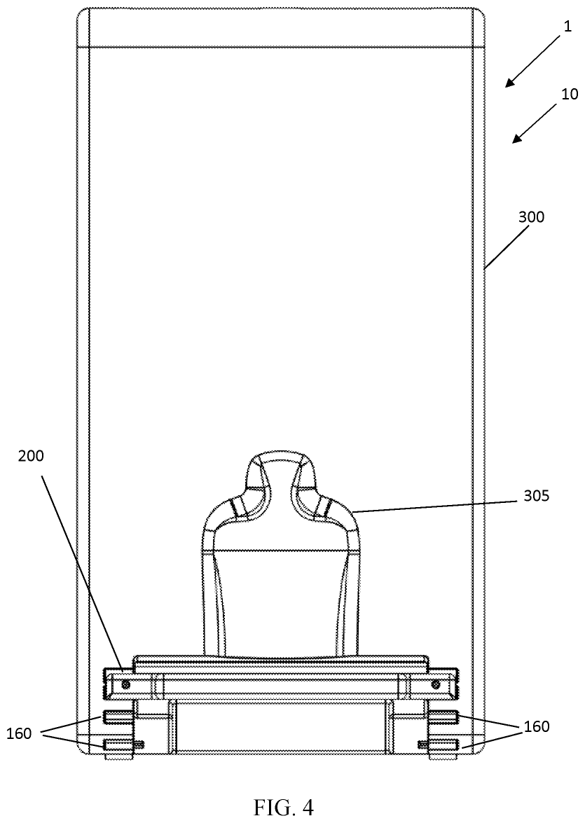

FIG. 4 is a top view of apparatus 1 with footpad 200 in the upright position 10.

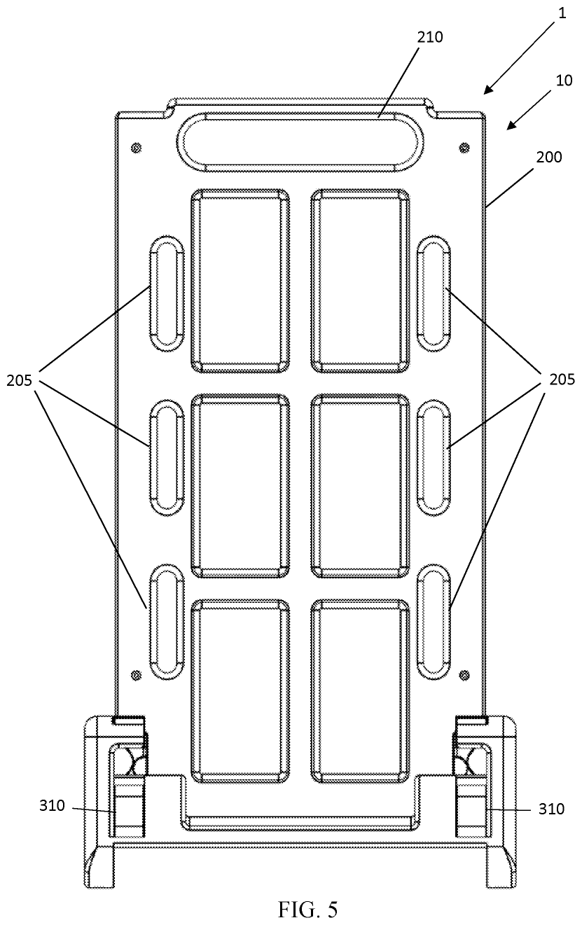

FIG. 5 is a rear view of apparatus 1 with footpad 200 in the upright position 10.

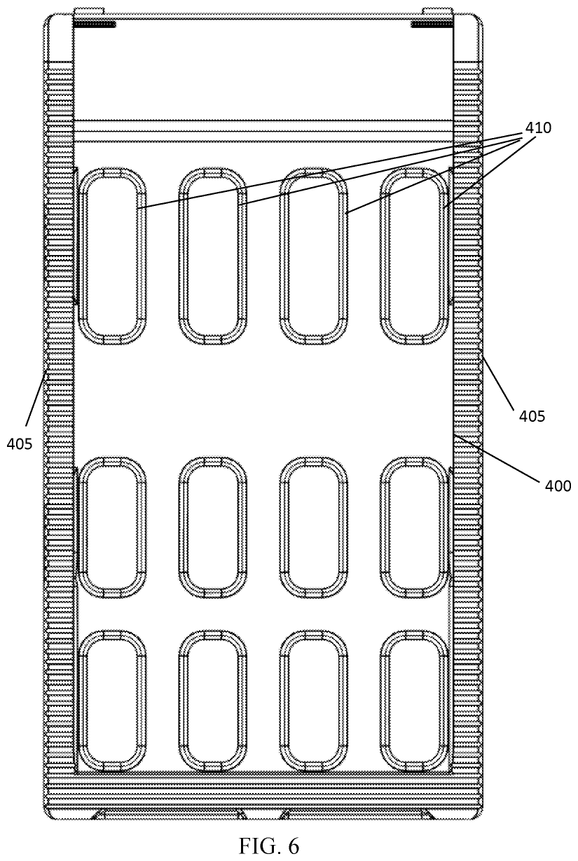

FIG. 6 is a bottom view of apparatus 1 showing base 100 with lower base 400.

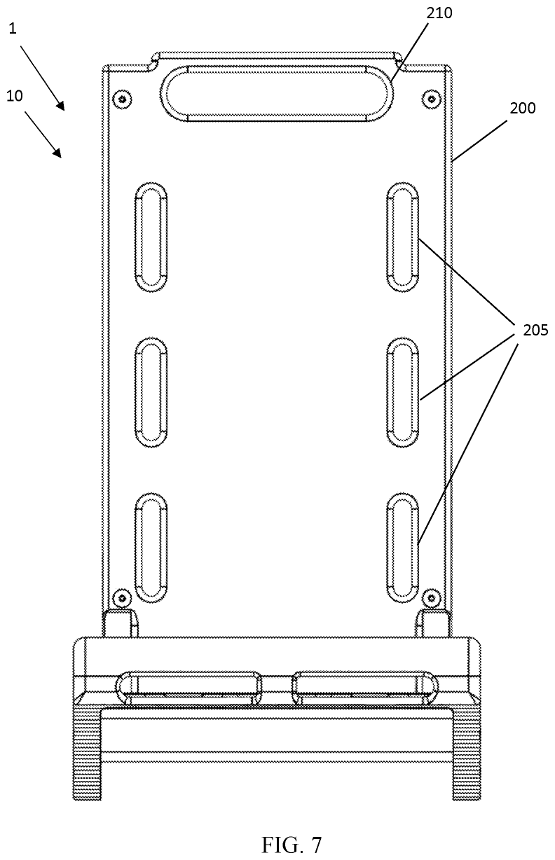

FIG. 7 is a front view of apparatus 1 showing footpad 200 in upright position 10.

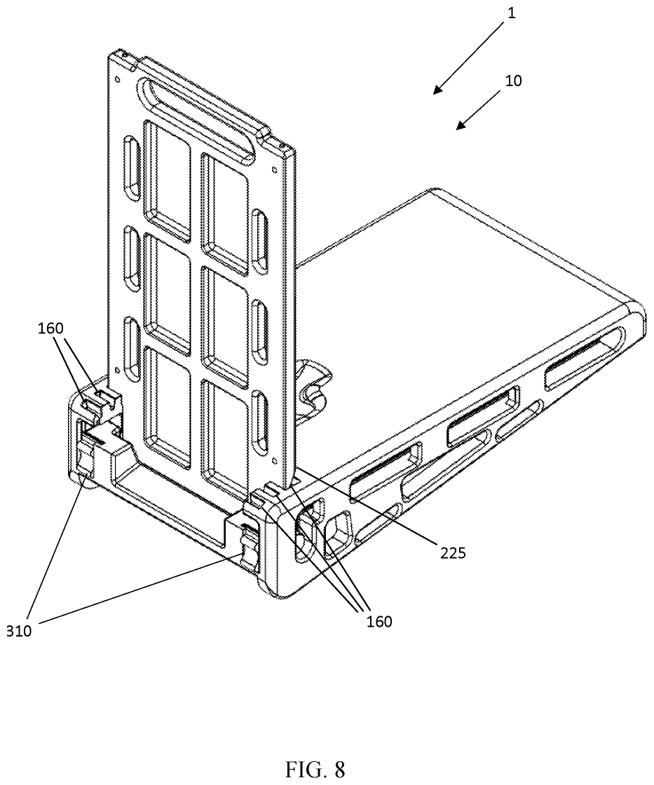

FIG. 8 is a rear isometric view of apparatus 1 with footpad 200 in upright position 10. Base 100 has four locking slots 160 on the left and the right side, although only three locking slots 160 are visible on either side in FIG. 8. The locking slots 160 receive positioning edge 225 of footpad 200. The user can select which set of locking slots 160 to use, such that footpad 200 can be placed in any number of positions ranging from forward-leaning, to perpendicular with calf plate 300, to rearward leaning, depending on the surgical or imaging procedure. Latch 310 is also visible in this figure. Latches 310 are on the left and right side of base 100, and allow footpad 200 to be held securely in place when it is in a stowed position.

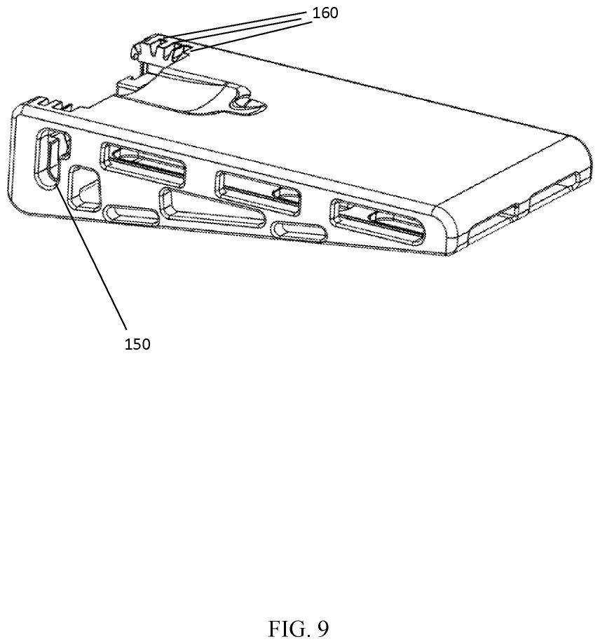

FIG. 9 shows a front isometric view of base 100 with footpad 200 removed for clarity.

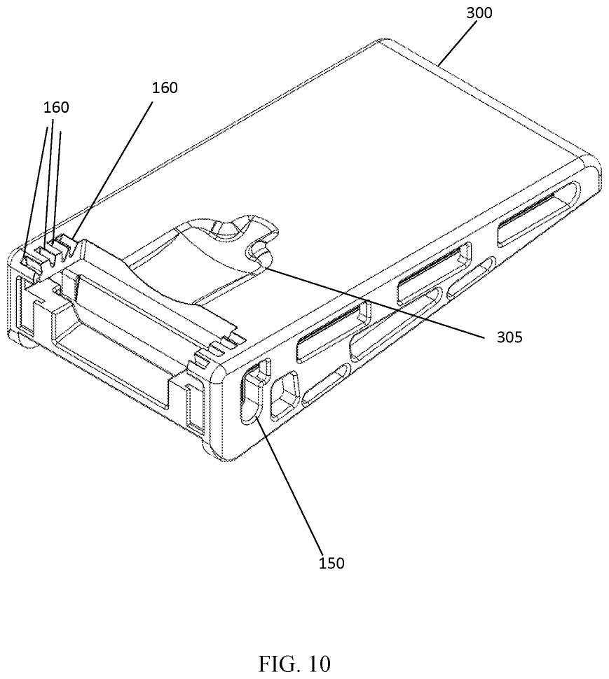

FIG. 10 shows a rear isometric view of base 100. In this view, the four locking slots 160 on each side of base 100 are visible. They receive positioning edge 225 to put footpad 200 in different angles relative to the patient's foot, as desired by the physician or technician.

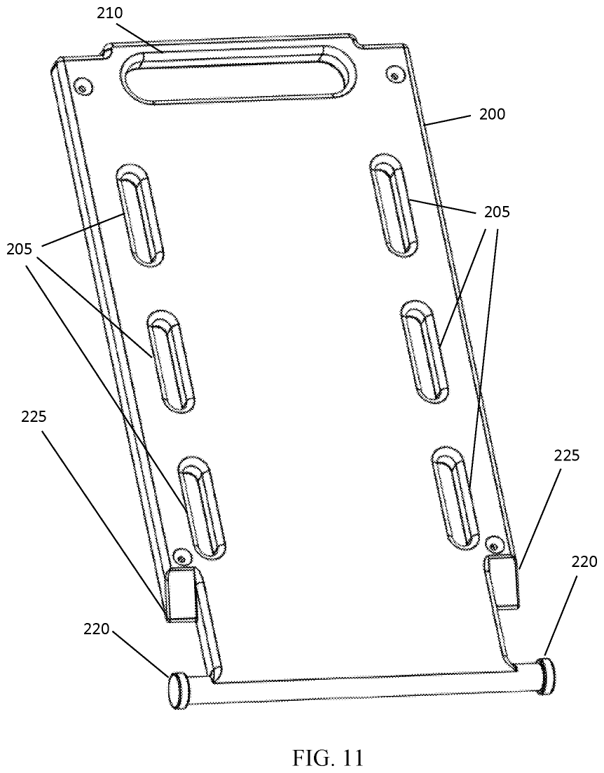

FIG. 11 shows a front isometric view of footpad 200. Lower pins 220 are integral to footpad 200, although they could also be separately attached pins. Lower pins 220 could also be rollers or casters to achieve even greater rolling ability. Lower pins 220 have a cap that will ride in a groove to be described later. Positioning edge 225 is located symmetrically on either side of footpad 200. Positioning edge 225 is also integral to footpad 200, or could alternatively be separate pieces mechanically attached. There are six footpad slots 205 located on footpad 200. Any number of footpad slots 205 could be used, but in this embodiment six are shown. Footpad slots 205 can be used for several functions. One function is that they can be used with straps to secure a patient's foot to footpad 200, so as to ensure good contact with footpad 200. Another use of footpad slots 205 is to allow easier cleaning and sterilization of footpad 200. A third use of footpad slots 205 is to mount surgical instruments, described later. Handle 210 also has several purposes. When apparatus 1 is being used in surgery or for imaging, handle 210 can be used to extract, position, and nest footpad 200 into base 100. When apparatus 1 is to be stored after use, then handle 210 can be used to carry apparatus 1.

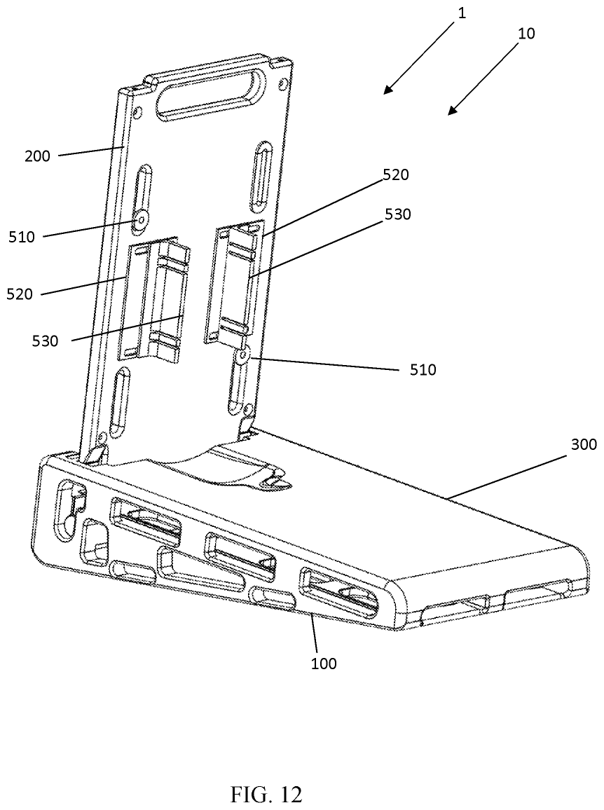

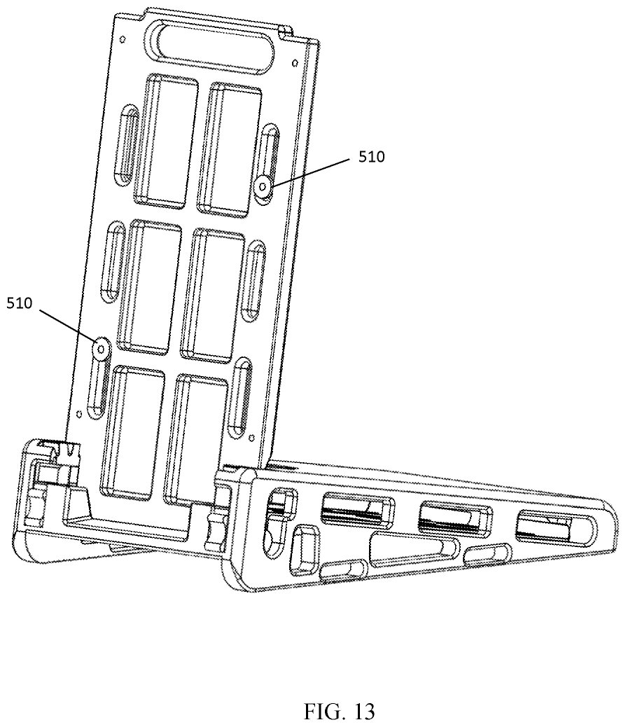

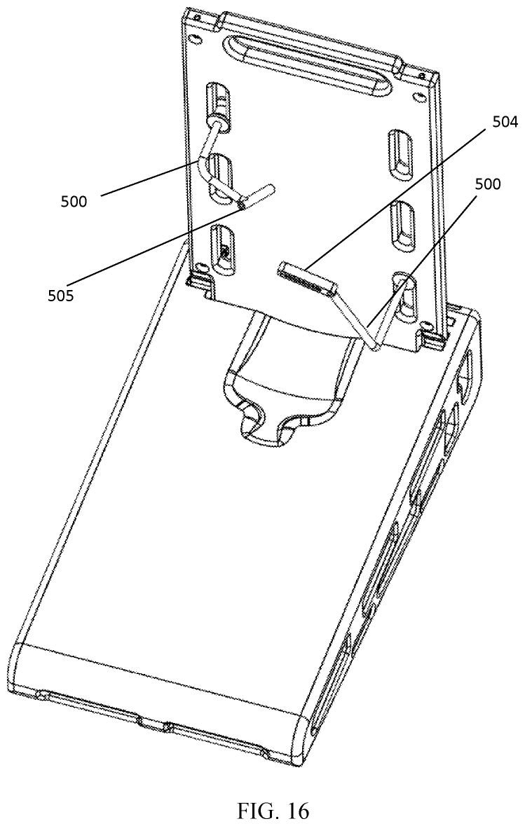

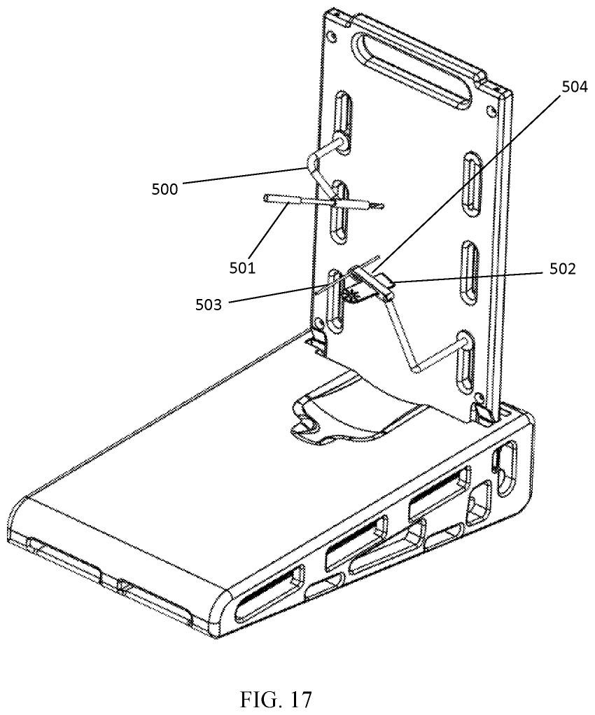

FIGS. 12 through 17 show numerous instrument mounting features such as grommets 510, support rails 520, slides 530, and instrument guides 500. Each instrument mounting feature may be formed in, attached to, received in, supported by, or otherwise coupled to the footpad. In some embodiments, an instrument mounting feature may be fixed relative to the footpad. In other embodiments, an instrument mounting feature may be movable, adjustable, repositionable, slidable, or removable relative to the footpad. FIG. 12 shows a front isometric view of apparatus 1 with grommets 510 and support rails 520 and slides 530 in place, and FIG. 13 shows a rear isometric view of the same embodiment. In this embodiment, grommets 510 consist of rubber cylinders that are positioned into the geometry of footpad slots 205. There is a hole in the center of each grommet 510 to receive an instrument, described later. Grommets 510 can be moved inside of each footpad slot 205, or relocated to other slots 205, or firmly attached to a slot 205, depending on the need of the user. There are many potential types of grommets 510 with different attachment mechanisms. Grommets 510 could be of a two-piece design, where one piece placed on the front side of footpad 200 screws into a second piece placed on the rear side to attach the grommet 510. Support rails 520 are made of a hard material in this embodiment, although many materials would suffice. Support rails 520 also attach to footpad slots 205 with pegs or screws that pass into and through slots 205. Support rails 520 have slots on them to receive slides 530. Slides 530 extend away from footpad 200 and are positionable within and upon support rails 520. Slides 530 can be used to constrain or hold a patient's foot in a particular orientation, as desired by the user.

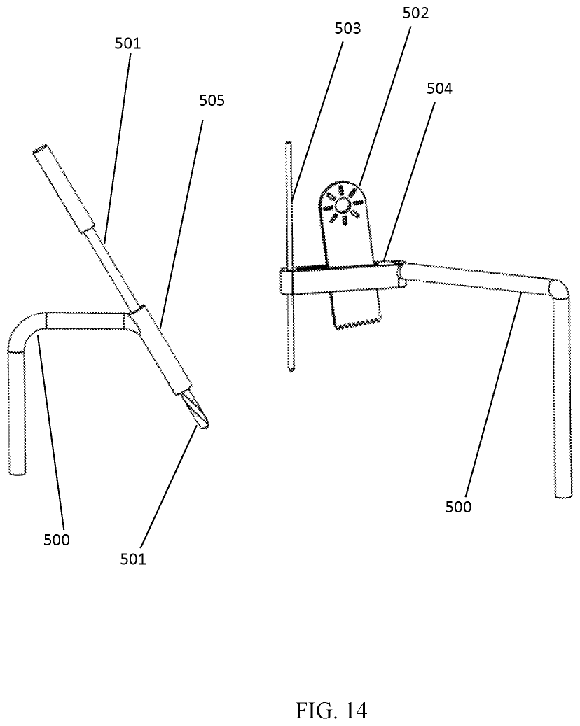

FIG. 14 shows instrument guides 500 and various other components. Instrument guide 500 is a device that inserts into grommet 510 on one end, and engages with a surgical instrument on the other. Instrument guide 500 can be flexible or stiff, depending on the application. Instrument guide 500 can also be designed such that it stows inside of footpad slot 205, such that instrument guide 500 can be left on the simulated weightbearing apparatus 1 for both upright position 10 and stowed position 5. Instrument guide 500 can also be articulated so that the physician can manipulate it into a desired position and then it maintains that position. With one end inserting into grommet 510, the other end of instrument guide 500 attaches to an instrument such as drill guide 505, saw slot 504, or any other type of instrument. An example instrument is drill guide 505 which is a tube that guides a drill bit 501 or locating pin 503 to contact patient tissue in a desired location. Similarly, saw slot 504 guides saw 502 to contact patient tissue in a desired location. Drill bit 501 is fluted and has a shaft, such that it can drill into tissue or bone. Locating pin 503 can either have a rounded tip, trocar tip, or pointed tip and can insert into tissue or bone to either mark a location, fit into another instrument, or insert into tissue. Saw 502 is serrated and designed to cut through tissue or bone in a controlled fashion. Working together, all the components shown in FIG. 14 can work together to allow a physician to perform surgical procedures with any number of instruments while a patient's foot is held in a simulated weightbearing position or any other position. The instrument guide 500 and instruments 501, 502, 503, 504, and 505 and any similar components can be part of kit packaged together, or packaged separately. The components can be provided sterile or non-sterile. They can be reusable or disposable, and made of metal, plastic or a combination of materials. They can be a standard surgical system or a minimally invasive system. In this way, a physician can use simulated weightbearing apparatus 1 with a kit of desired instruments to perform surgery and imaging.

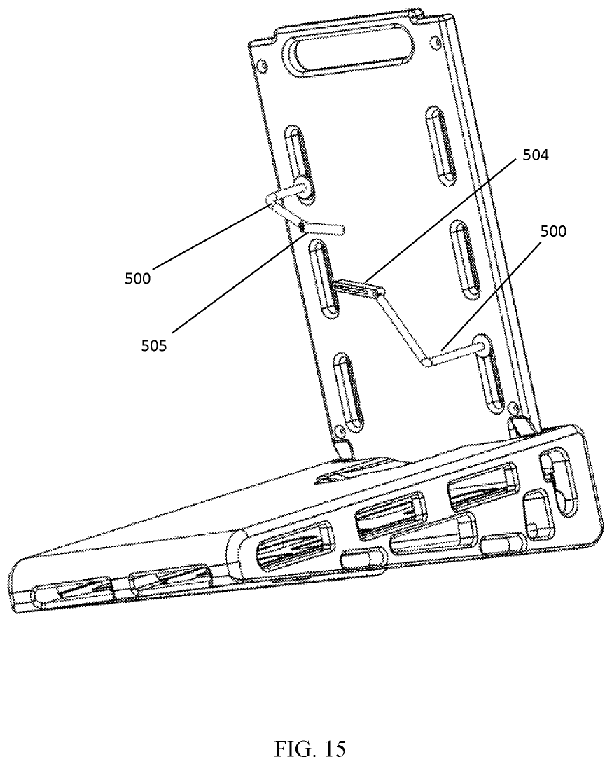

FIGS. 15, 16, and 17 show instrument guides 500, drill guide 505, drill bit 501, saw slot 504, saw blade 502, and locating pin 503 all being used on simulated weightbearing apparatus 1 with footpad 200 in upright position 10. Patient leg 75 and foot 80 are not shown for clarity. The instrument guides 500 connect to footpad 200 via grommets 510, which pass through footpad 200 and provide an anchorage point.

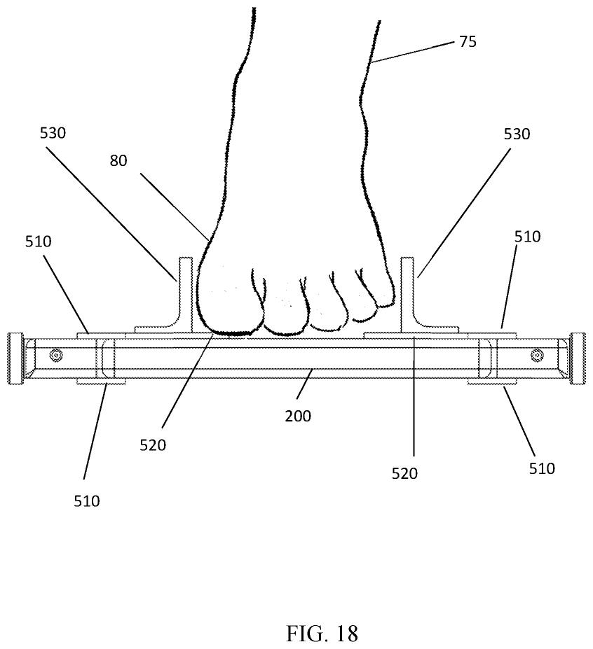

FIG. 18 shows a view looking down from the top of footpad 200. In this view, patient leg 75 is visible along with representative patient foot 80. Grommets 510 are visible that could be used to hold instrument guide 500. Support rails 520 are visible, along with slides 530. Support rail 520 is a device that attaches to footpad 200 as mentioned previously. Slide 530 moves across support rail 520 to contact, squeeze, constrain, or stabilize foot 80 in a location desired by a user. In this way, slides 530 can position foot 80 upright or shift foot 80 to an outward-leaning position or inward-leaning position. The foot 80 may be held firmly to allow reliable and repeatable imaging or to maintain a stable position for surgery. Slides 530 can be made of a rigid material but covered with a cushion if desired.

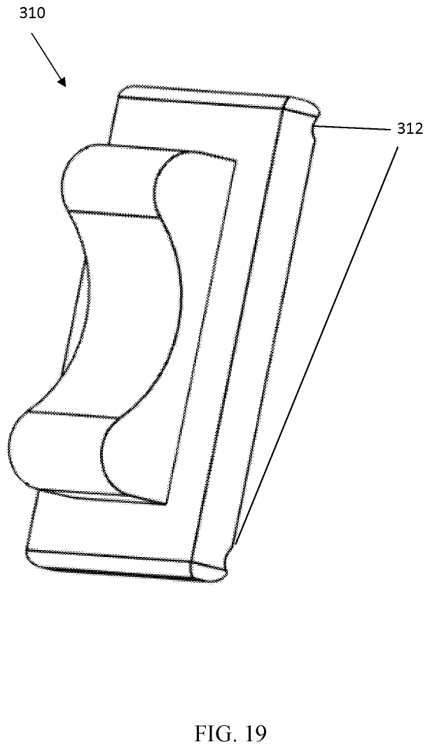

FIG. 19 shows latch 310. Latch 310 allows footpad 200 to be held in a stowed position and simulated weightbearing apparatus 1 to be carried by handle 210, without the footpad 200 swinging loose from base 100. Latch 310 is designed to slide in latch slot 170. Latch indentations 312 in latch 310 move along latch slot 170 until they engage latch slot bumps 171. The nestling of latch slot bumps 171 in latch indentations 312 holds latch 310 in place, but can be overcome by a user when desired to release the latch 310.

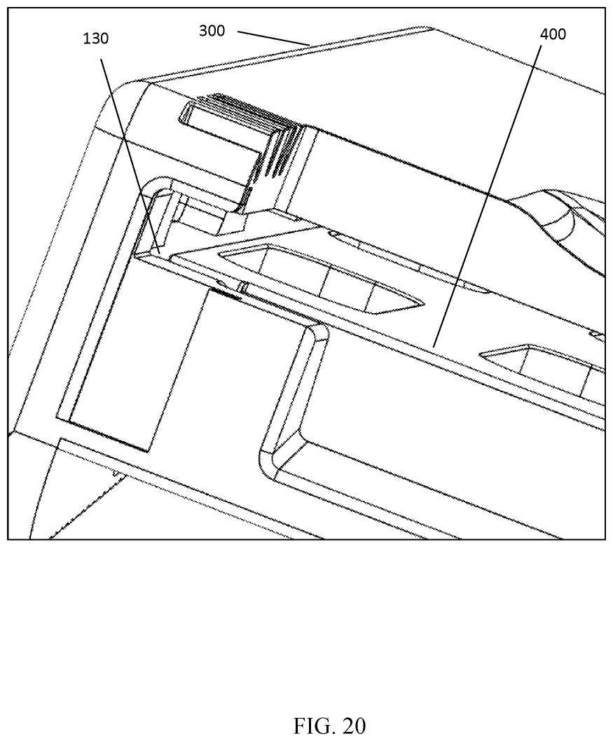

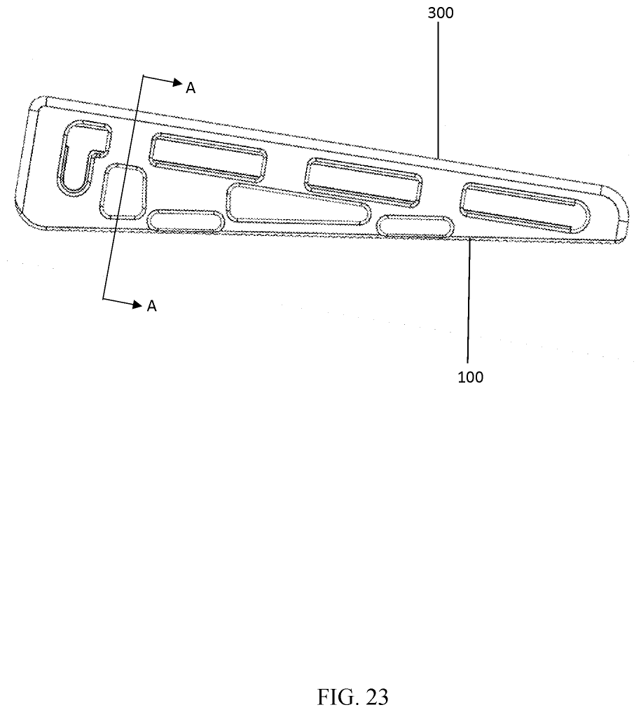

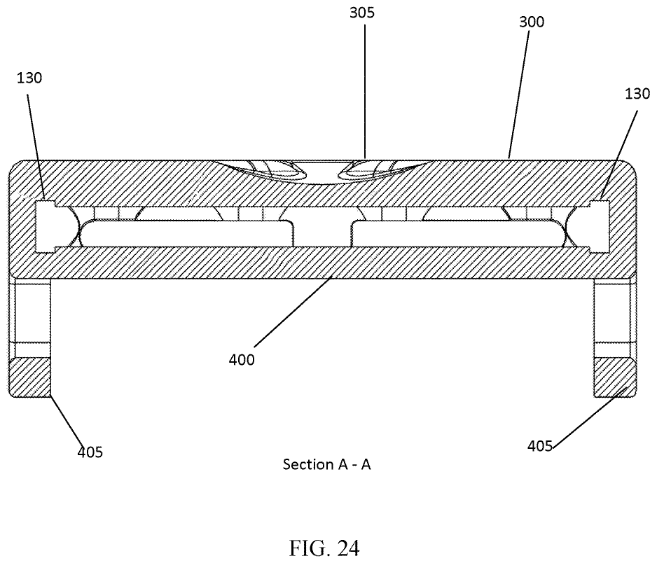

FIG. 20 shows an isometric rear close-up view of the base 100. Footpad 200 is removed for clarity. In this view, a portion of base slot 130 is visible. The lower pins 220 on either side of footpad 200 slide through base slot 130. There are two base slots 130, one on either side of base 100. The base slots 130 are normally hidden from view and are best visualized in FIGS. 23 and 24. FIG. 23 shows a section A-A through base 100 that passes through base 100 and calf plate 300 in the middle of heel cup 305. FIG. 24 shows a clear view of base slot 130 on each side of base 100 in this section. This base slot 130 continues along the length of base 100 until it turns downward at corner slot 150. The entire length of base slot 130 is shaped to allow lower pins 220 to slide smoothly. In the event lower pin 220 is replaced with a caster, roller, or wheel, then it would still roll through base slot 130.

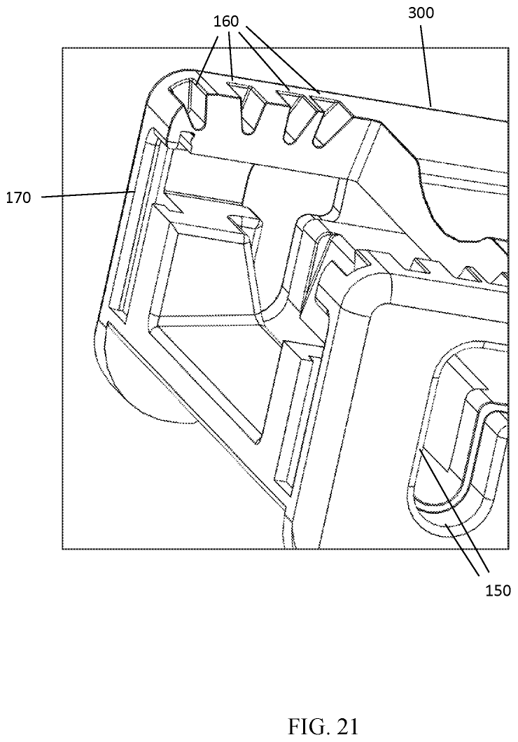

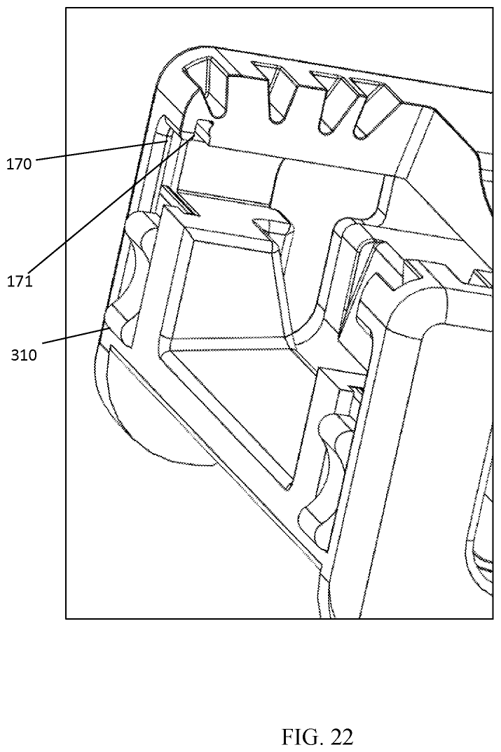

FIGS. 21 and 22 also show a close-up isometric view of the rear of base 100 with footpad 200 removed for clarity. In FIG. 21, latch 310 is removed and in FIG. 22 latch 310 is visible. In these figures, latch slot 170 in base 100 is visible. There are symmetrical latch slots 170 on each side of base 100. An identical latch 310 fits in each latch slot 170, and latch 310 slides up and down in latch slot 170. As latch 310 moves up, latch slot bump 171 engages with latch indent 312 to hold latch in an upper position. In this upper position, latch 310 prevents footpad 200 from moving along base slot 130, so that apparatus 1 can be carried by handle 210 and the parts do not swing loose from each other. With latch 310 in the upper position, apparatus 1 is in its storage position.

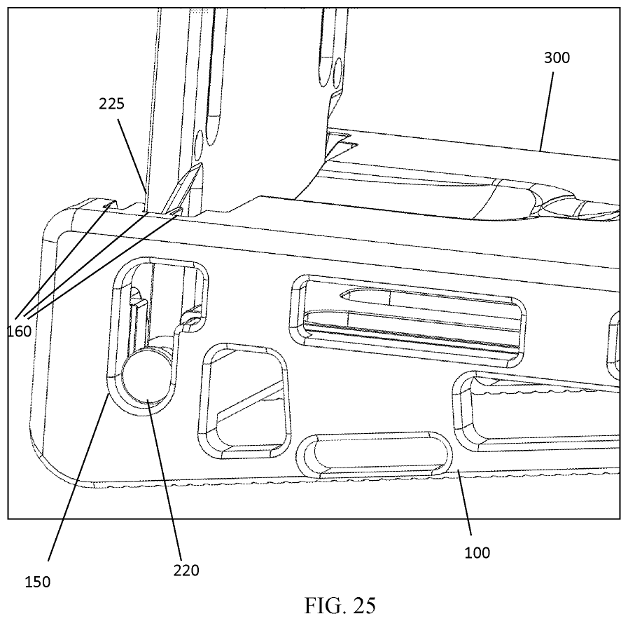

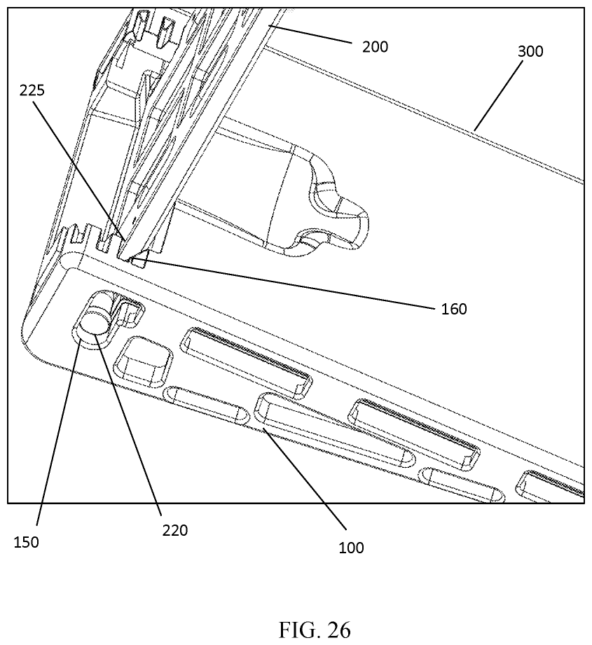

FIGS. 25 and 26 both show a close-up left side and top left isometric view of the rear of base 100 with footpad 200 in position 10. Now it can be seen that positioning edge 225 is engaged in the second locking slot 160 on the top of base 100. Multiple locking slots 160 are visible from this view, and the user can choose which locking slot 160 is appropriate for the imaging or surgical procedure. Lower pin 220 of footpad 200 has descended to the bottom of corner slot 150 in base 100. In this configuration, when a patient's foot presses against footpad 200, a force is created that is resisted by a couple moment formed between positioning edge 225 engaged in locking slot 160 and lower pin 220 resting in corner slot 150 of base 100. The flat rear side of positioning edge 225 makes a sturdy connection in locking slot 160. Note that positioning edge 225, locking slot 160, lower pin 220, and corner slot 150 are all symmetric on the right and left sides of the simulated weightbearing apparatus 1.

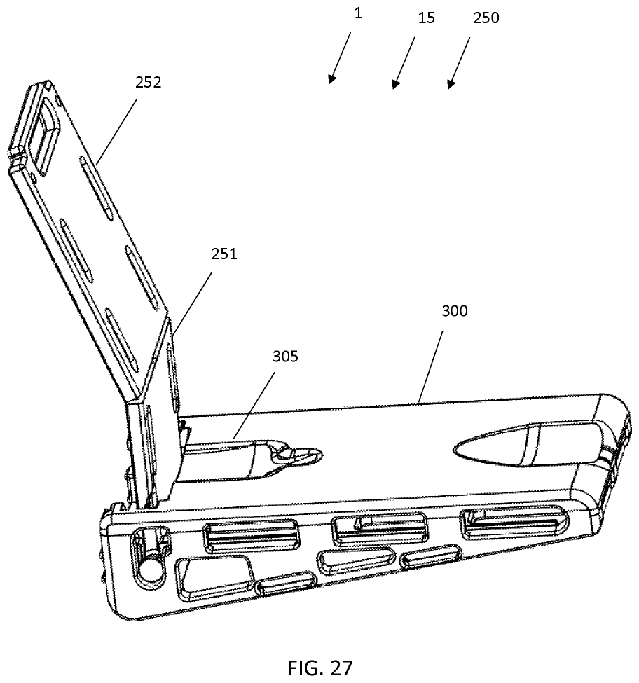

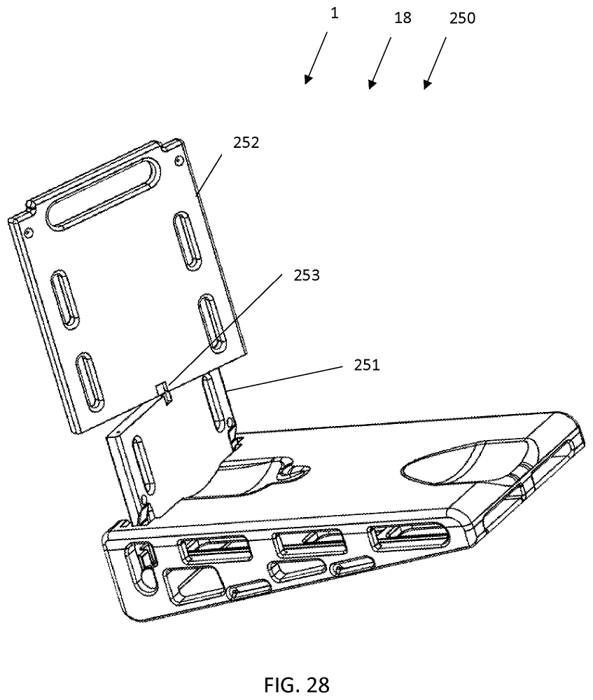

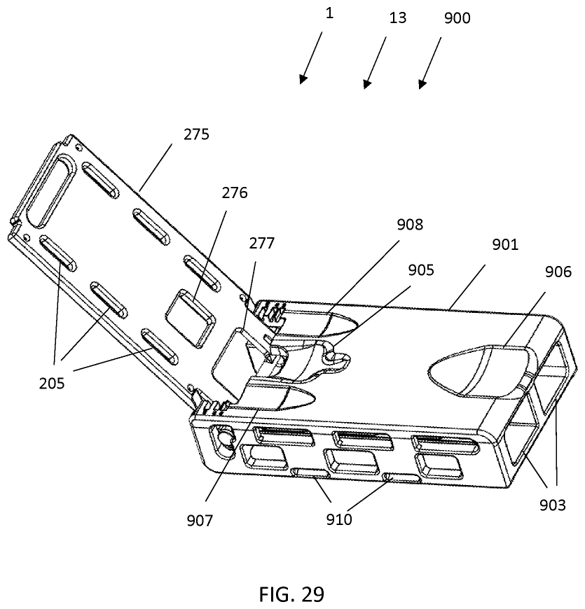

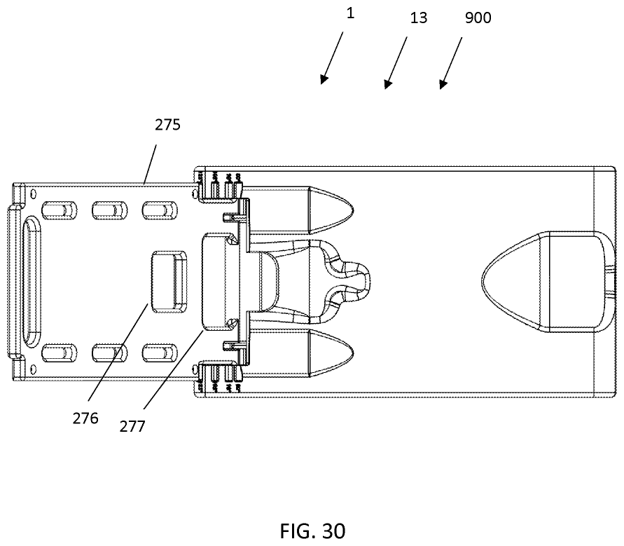

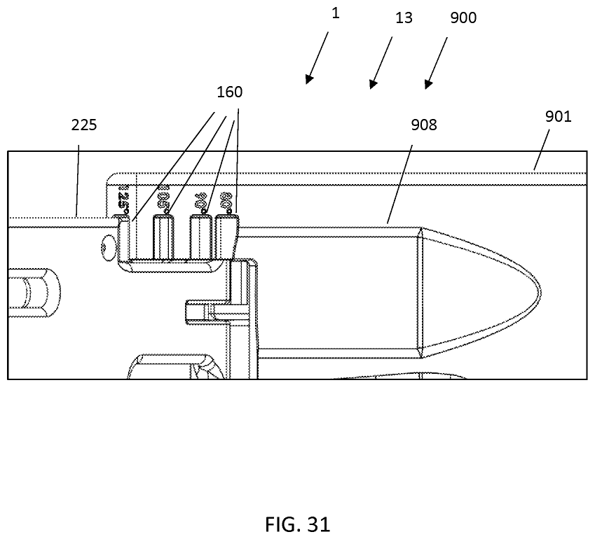

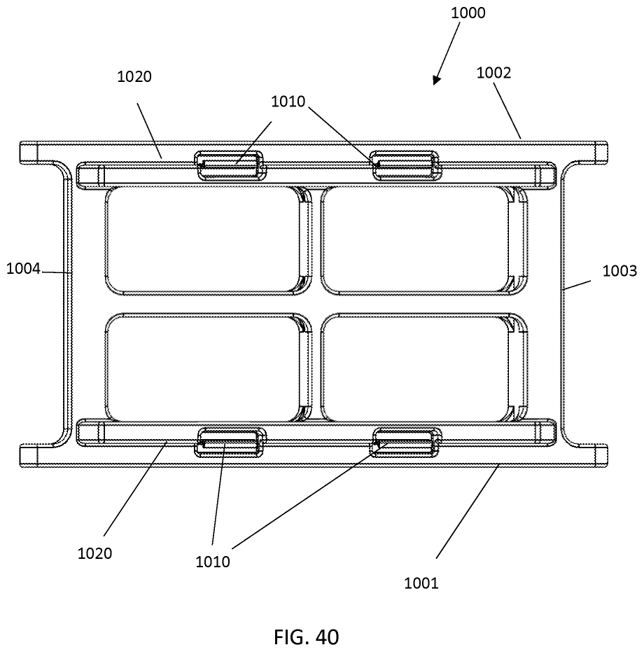

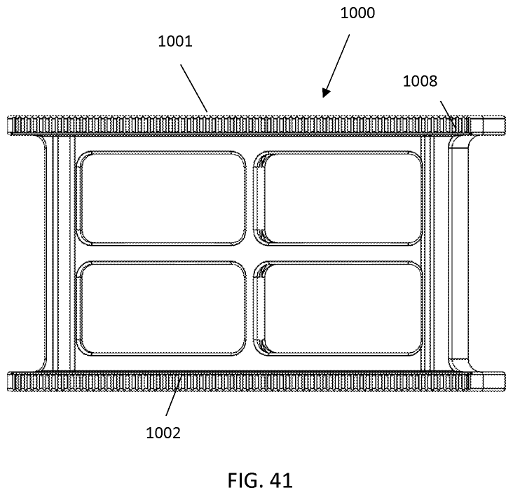

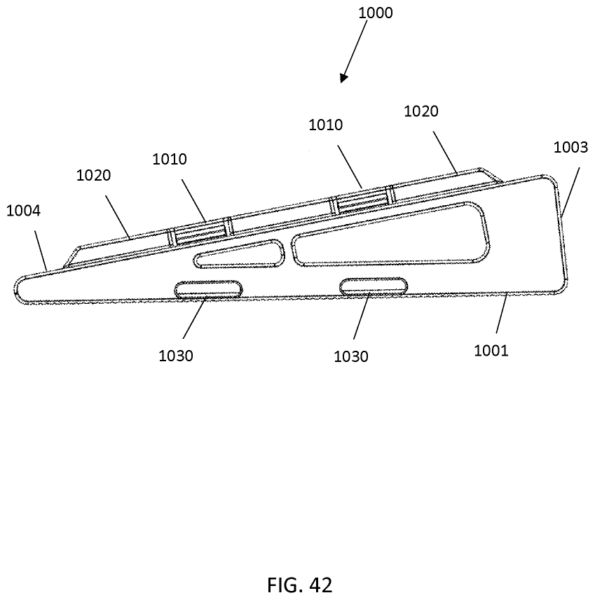

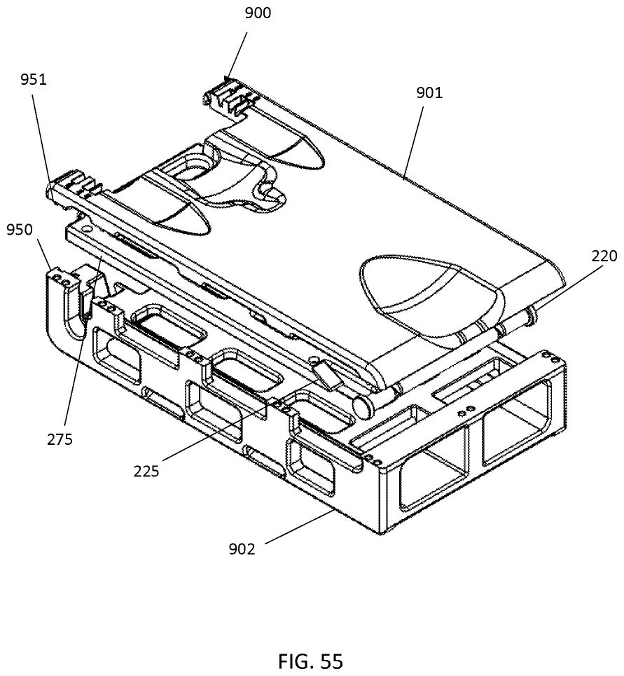

FIGS. 27 and 28 display a first embodiment of the simulated weightbearing apparatus 1. Major components include base 100, footpad 250, and calf plate 300 which is the top surface of base 100. Footpad 250 is an assembly of three parts. Upper footpad 252 and lower footpad 251 are connected by pivot 253. Pivot 253 can be a mechanical hinge, living hinge, axle, ball bearing, or any other mechanism that allows upper footpad 252 and lower footpad 251 to move independently of each other with one, two, or three degrees of freedom. By positioning upper footpad 252 and lower footpad 251 in different orientations, a physician can allow footpad 250 to contact a patient's foot in various positions. Footpad position 15 is shown in FIG. 27 with upper footpad 252 angled in one plane relative to lower footpad 251. Footpad position 18 is shown in FIG. 28 with upper footpad 252 twisted about a longitudinal axis relative to lower footpad 251. Although this embodiment shows only a single pivot 253, any number of pivots could be included in the design to create more complex positioning and orientation of the patient's foot when it engages the footpad 250.
FIGS. 29 through 56 display a second embodiment of a simulated weightbearing apparatus. FIG. 29 is an isometric view of apparatus 1 with the footpad 275 in the most angled position 13. In angled position 13, a patient's foot would not be at a 90 degree angle with the patient's leg. A physician might use this angled position for surgery on a patient's foot or for imaging. Base 900 has numerous features shown. Calf plate 901 is the upper piece of base 900. Heel cup 905 is centered at the distal end of base 900, where a patient's heel could comfortably rest during imaging or surgery. Left heel cup 907 and right heel cup 908 are alternate heel cups that could receive a patient's heel, in the event the physician wanted to position the patient's foot at a different location on the footpad 275. For example, a physician might desire to position a patient's right foot in the left heel cup 907 so that the hallux is on the outside of simulated weightbearing apparatus 1. Calf cup 906 is a rounded area on calf plate 901 that allows a patient's leg and calf to rest more comfortably. Strap slots 910 allow for base 900 to be secured to an exam bed or to wedge 1000, which will be discussed later. There are four strap slots 910, but there could be any number of strap slots 910 to accommodate as many straps as desired. Longitudinal slots 903 extend from one end of base 900 to the other. Two purposes of longitudinal slots 903 are to reduce the weight of base 900, and make it easier to access internal surfaces for cleaning. Alternatively, longitudinal slots 903 could also be used to route straps around the exam bed or the patient's leg to stabilize the weightbearing apparatus 1. Footpad 275 is shown with access openings that allow the physician to access the plantar surface of the patient's foot. Footpad 275 includes upper access 276 and lower access 277. Upper access 276 and lower access 277 are through holes in footpad 275 that allow a physician to access the bottom surface of a patient's foot when the patient's foot is positioned against footpad 275. For example, upper access 276 might accommodate access to the bottom of a patient's midfoot and toes while lower access 277 might allow access to the bottom of the patient's hindfoot or calcaneal area. Pad slots 205 allow a physician to secure a strap around the patient's foot, to hold it tightly to footpad 275. Although six pad slots 205 are shown, there could be more or less depending on foot size and a surgeon's needs.
FIGS. 30 and 31 show a top view of weightbearing apparatus 1 in angled position 13. FIG. 31 is a close-up view of the right rear corner of base 900 that is shown in FIG. 30. Positioning edge 225 is an edge on footpad 275 that engages with locking slots 160 that are part of base 900. There is a symmetrical positioning edge 225 on the left and right side of footpad 275, and both positioning edges 225 engage with locking slots 160 at the same time. Footpad 275 can be oriented to different angles relative to base 900, and secured in place when positioning edge 225 on each side is inserted into one of the locking slots 160. There are multiple locking slots 160 that correspond to different angulations of footpad 275, depending on physician preference.

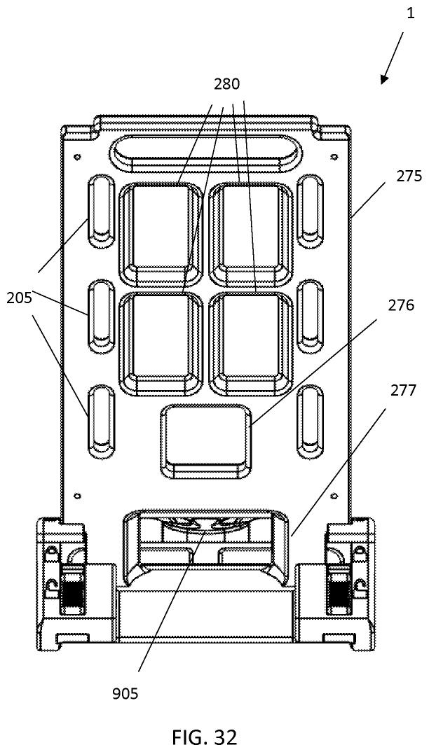

FIG. 32 shows a rear view of simulated weightbearing apparatus 1 in angled position 13. Pad slots 205 can be seen and are thru holes in footpad 275 to allow straps to be placed around a patient's foot, or pad slots 205 can also serve as attachment points for surgical instrumentation. Recess cavities 280 are also visible on the back side of footpad 275. Recess cavities 280 do not go all the way through footpad 275, and can serve several purposes. Recess cavities 280 allow the mass of footpad 275 to be reduced if desirable. Recess cavities 280 also allow for a different amount of material to appear behind a patient's foot during imaging, which can show up as faint straight lines on the x-ray. Thus, recess cavities 280 can create an imaging pattern or grid to assist the surgeon in measuring distances or aligning the bones of the foot. The quantity, shape, and location of recess cavities 280 can vary depending on the requirements of the physician or hospital. Upper access 276 and lower access 277 are visible from the back of footpad 275. The heel cup 905 is also partially visible on base 900 through lower access 277.

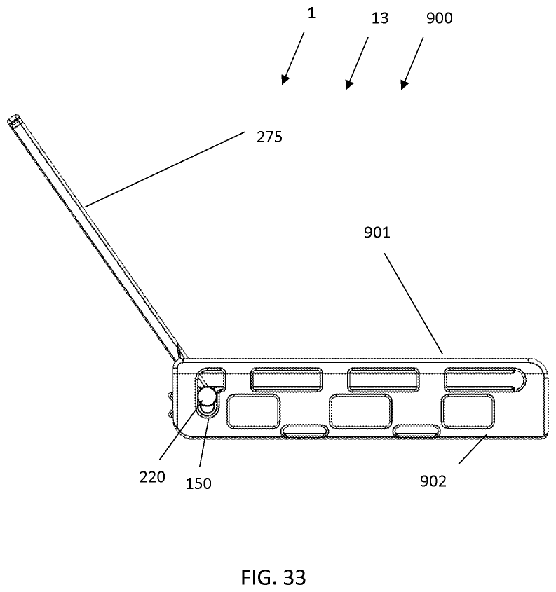

FIG. 33 is a side view of simulated weightbearing apparatus 1 with footpad 275 deployed in angled position 13. Lower pin 220 is shown moving into corner slot 150 of base 900. When lower pin 220 is in corner slot 150 the footpad 275 becomes stable relative to base 900. Base 900 is an assembly of two pieces. Calf plate 901 is the top piece, and attaches to lower base 902. Footpad 275 slides between calf plate 901 and lower base 902 when it is in the stowed position for storage or keep it temporarily out of the way during surgery.

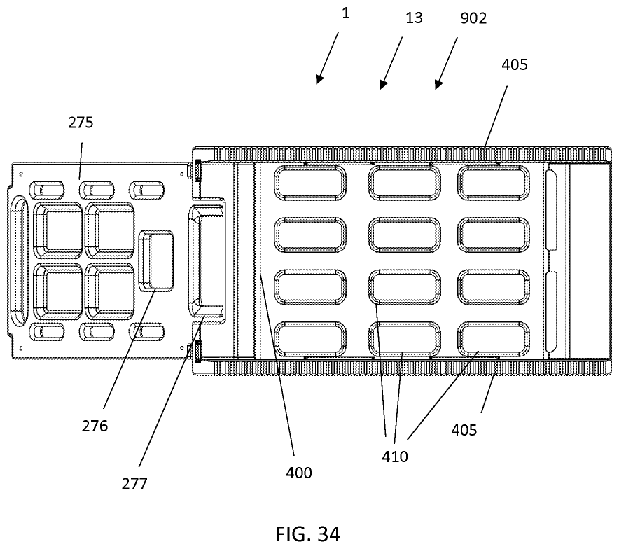

FIG. 34 is a bottom view of simulated weightbearing apparatus 1 with footpad 275 in angled position 13. Lower base 902 is visible from this view. Lower base 902 has bottom holes 410 that allow for easier access of cleaning and steam sterilization for simulated weightbearing apparatus 1. The quantity, shape, and location of bottom holes 410 can vary depending on requirements, but in this embodiment twelve are shown. Grips 405 are located on the bottom surface of lower base 902. Grips 405 provide better friction on a bed or imaging surface, so that apparatus 1 resists sliding.

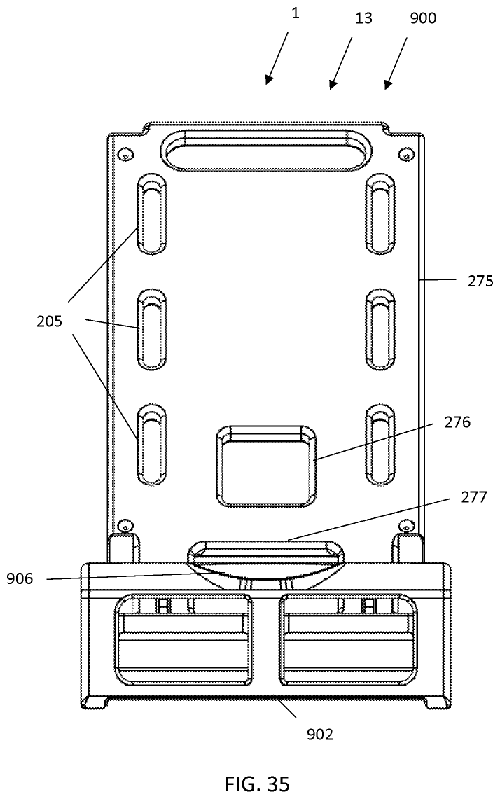

FIG. 35 is a front view of simulated weightbearing apparatus 1 with footpad 275 in angled position 13. Calf cup 906 is visible from this view, located on calf plate 901. Lower base 902 is also visible.

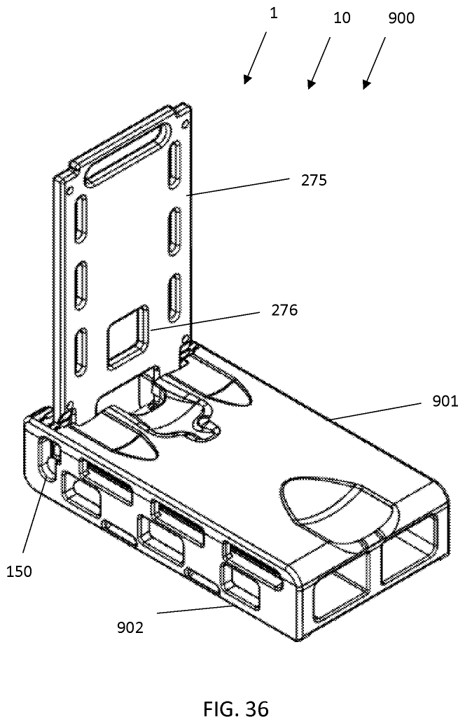

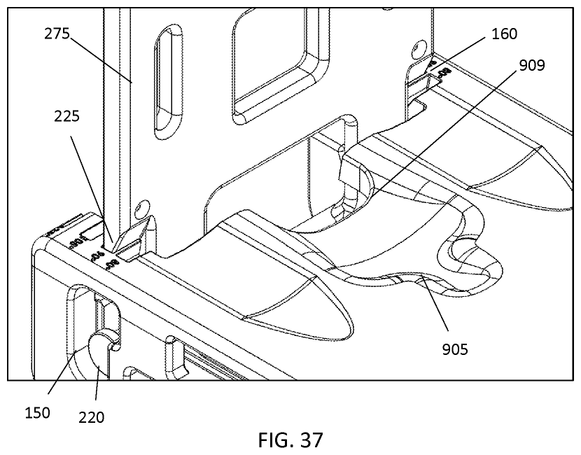

FIGS. 36 and 37 show an alternative configuration for simulated weightbearing apparatus 1. Footpad 275 is in the upright position 10 in both FIGS. 36 and 37. Upright position 10 represents approximately a 90 degree position of footpad 275 relative to calf plate 901, and allows the physician to place a patient's foot in a 90 degree position. FIG. 37 shows an enlargement of how footpad 275 interfaces with calf plate 901 and base 900. Positioning edge 225, on both the left and right side of footpad 275, engage with locking slots 160 in the 90 degree position. Locking slots 160 can be marked via engraving, laser etching or pad printing to show specific angles if desired. Lower pin 220 is fully engaged in the corner slot 150 of lower base 902. When a patient's foot presses against footpad 275, a couple moment is created between two forces. The first force is at the positioning edge 225 interacting with locking slots 160, and the second force is at the lower pins 220 in the corner slot 150 of lower base 902. This couple moment resists the pressure of a patient's foot. FIG. 37 also displays cut out 909 in calf plate 901. Cut out 909 is an opening in calf plate 901 that coincides with lower access 277 to allow for physician access to the rear of a patient's foot.

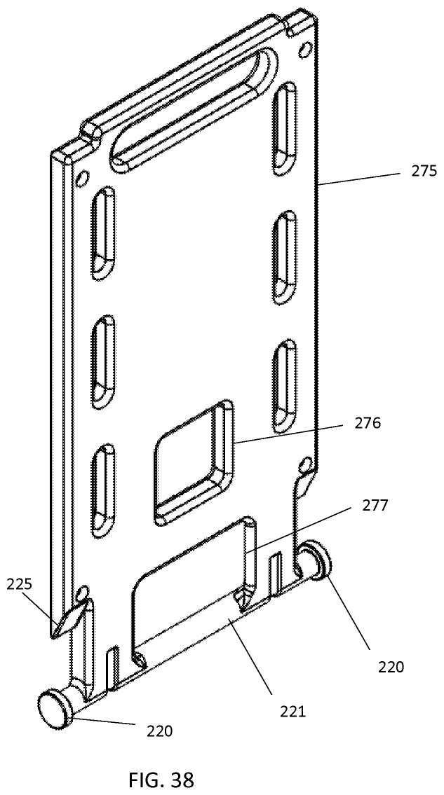

FIG. 38 shows an isometric view of footpad 275. Lower pins 220 are visible on both sides of footpad 275, and are connected to each other by shaft 221. In this embodiment, all of footpad 275 is made from an integral piece of material. However, it is also possible that lower pins 220 and shaft 221 could be separate assembles and thus rotate independently of footpad 275. A designer could use this feature such that lower pins 220 roll into corner slot 150 instead of sliding.

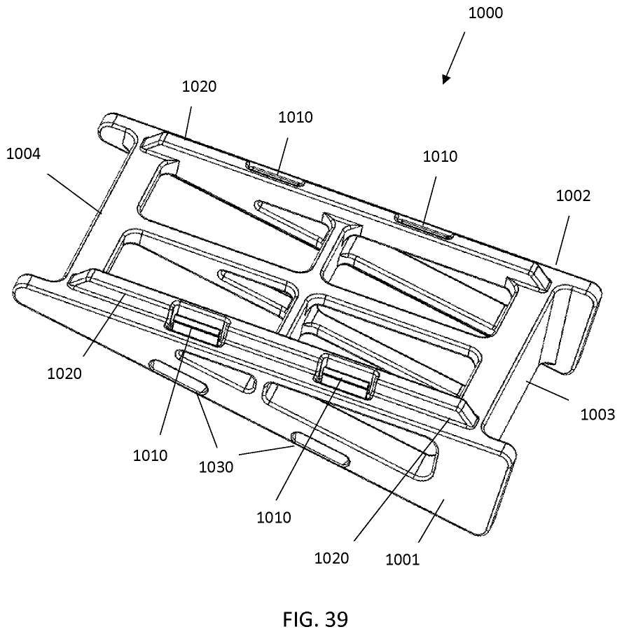

FIGS. 39-42 show isometric, top, bottom, and side views of wedge 1000. Wedge 1000 will be used with simulated weightbearing apparatus 1, but wedge 1000 is a separate device. Wedge 1000 has front end 1004, rear end 1003, left side 1001, and right side 1002. There are two rails 1020 on the top surface of wedge 1000. Multiple projections 1010 are present on rails 1020. Wedge slots 1030 are located near the bottom of wedge 1000. Projections 1010 are living hinges that can deflect and snap into strap slots 910 or wedge slots 1030, or that can snap onto the bottom edge of wedge 1000. Projections 1010 deflect when apparatus 1 or wedge 1000 is placed on a second wedge 1000, and then projections 1010 snap into strap slots 910 or wedge slots 1030. Rails 1020 of one wedge 1000 fit between the bottom edges of a second wedge 1000 to provide stability. Wedge grips 1008 are roughened surfaces or ridges to resist sliding of the bottom of wedge 1000. Although two wedges 1000 are shown in several examples, the system may use one wedge, two wedges, or more than two wedges.

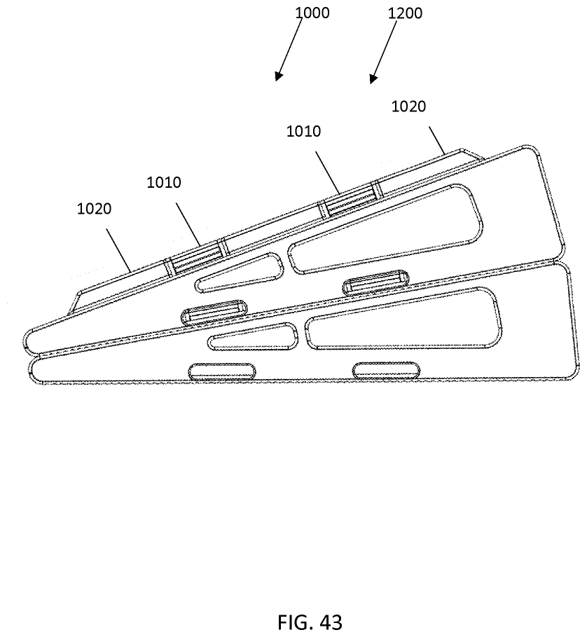

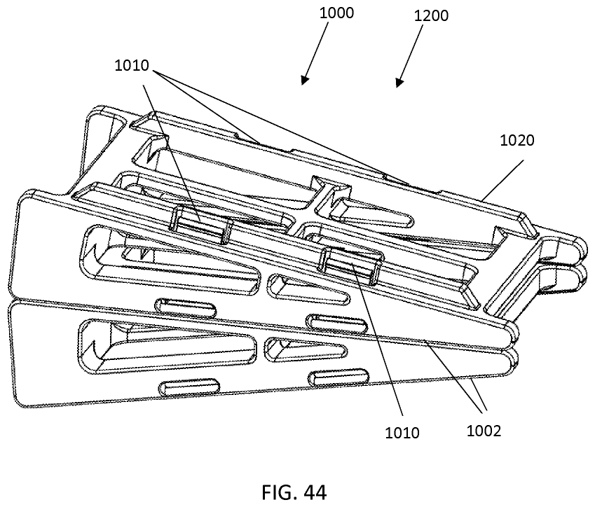

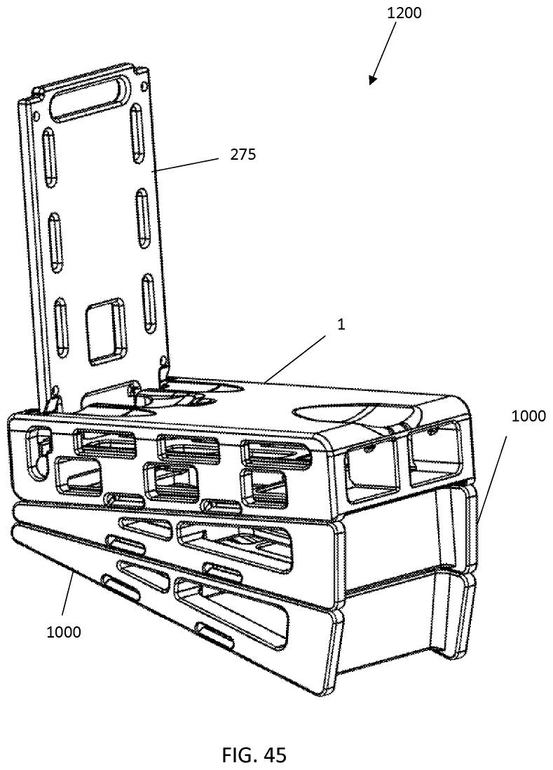

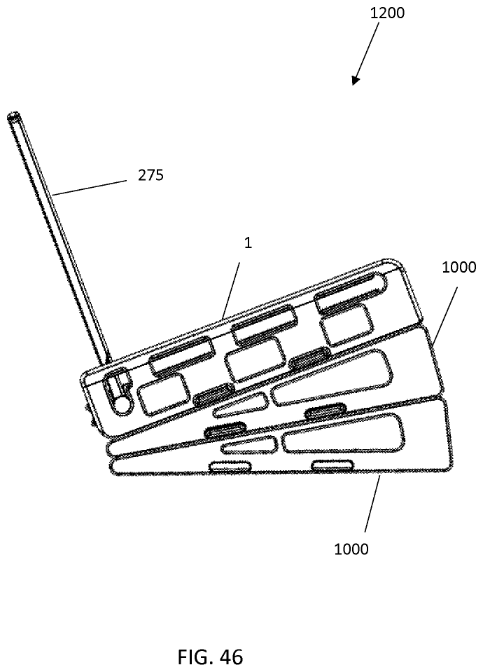

FIGS. 43 and 44 show how two wedges 1000 can be placed on top of each other in a first arrangement, in inclined position 1200. In inclined position 1200, the wedges are aligned such that it steepens the angle of the wedge. Projections 1010 on the lower wedge 1000 engage with wedge slots 1030 on the upper wedge 1000 to make the two wedges secure and stable. In addition, rails 1020 on the lower wedge 1000 fit between the bottom edges of the upper wedge 1000 yielding additional stability. FIG. 45 then shows two wedges 1000 in inclined position 1200 with simulated weightbearing apparatus 1 placed on top. Projections 1010 on the upper wedge 1000 snap into strap slots 910 on apparatus 1. In this configuration, a patient's leg can be bent at the knee and then the foot placed against the footpad 275 of apparatus 1. Thus, the purpose of wedge 1000 is to allow for more orientations of patient's foot and leg when using simulated weightbearing apparatus 1. FIG. 46 shows the same configuration of apparatus 1 and wedges 1000 from a side view.

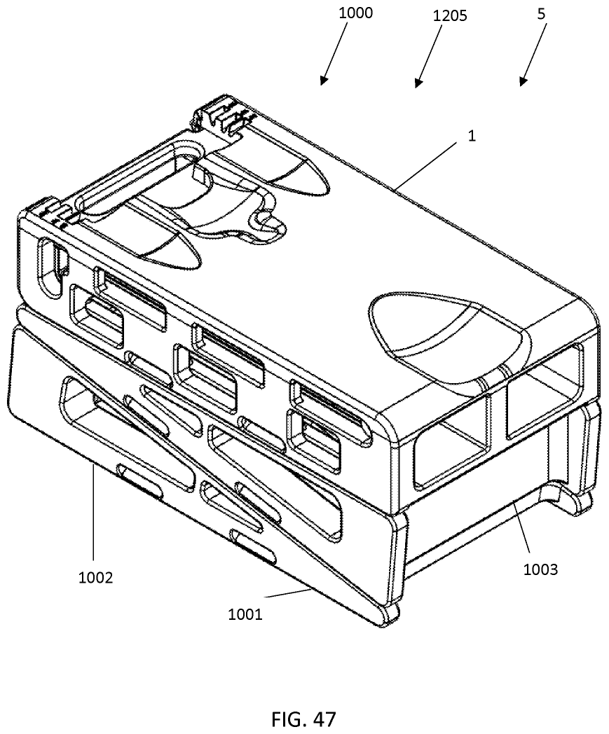

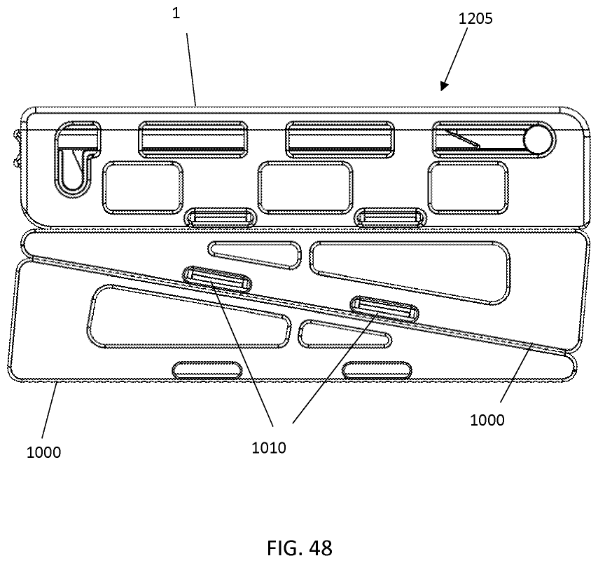

FIGS. 47 and 48 show an isometric and side view, respectively, of simulated weightbearing apparatus 1 with footpad 275 in stowed position and two wedges 1000 in a second arrangement, which is elevated position 1205. Elevated position 1205 has the two wedges 1000 oriented in opposite directions, such that the wedges offset each other and thus apparatus 1 is raised vertically without substantially inclining the apparatus. In this scenario, a physician or technician could elevate a patient's foot for surgery or imaging. For example, it is commonly a problem for lateral images of the foot to have the opposing foot in the way of a clean image. The use of the devices in FIGS. 47 and 48 solve this problem by elevating the foot that requires imaging. The inclined position 1200 may be referred to as an inclined orientation, and the elevated position 1205 may be referred to as an elevated orientation. Wedges 1000 may be stackable in different arrangements, including the inclined position 1200 and the elevated position 1205.

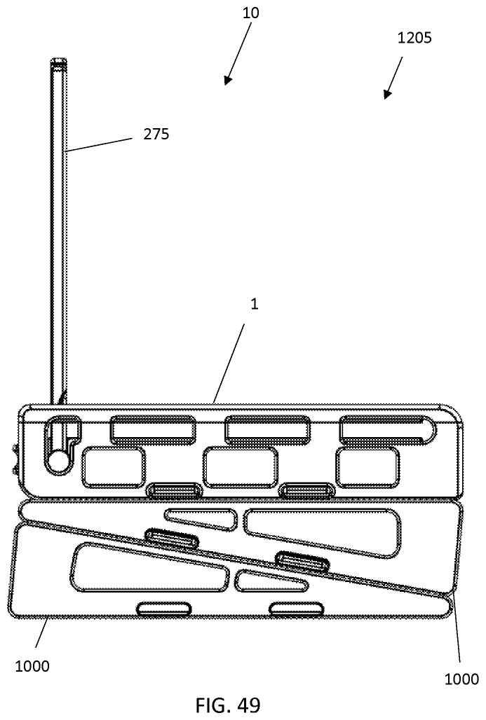

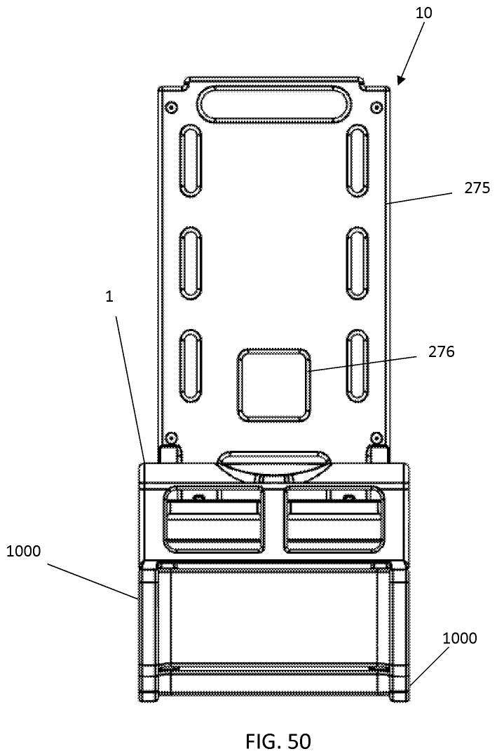

FIGS. 49 and 50 show different views of simulated weightbearing apparatus 1 on top of two wedges 1000, with the two wedges 1000 in elevated position 1205. Footpad 275 is in the upright position in these figures.

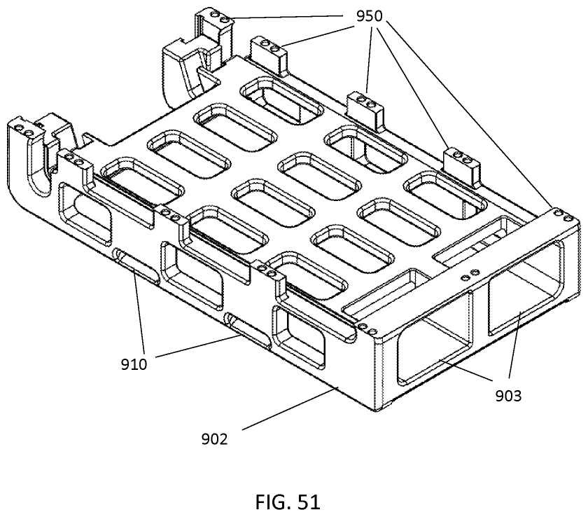

FIG. 51 shows an isometric view of lower base 902. Lower base 902 features previously mentioned strap slots 910 and longitudinal slots 903. Lower base 902 also has a series of peg receptacles 950. In this embodiment, there are eleven pairs of peg receptacles 950 shown, but there could be more or less depending on design requirements. The purpose of peg receptacles 950 are to align and provide an attachment for calf plate 901.

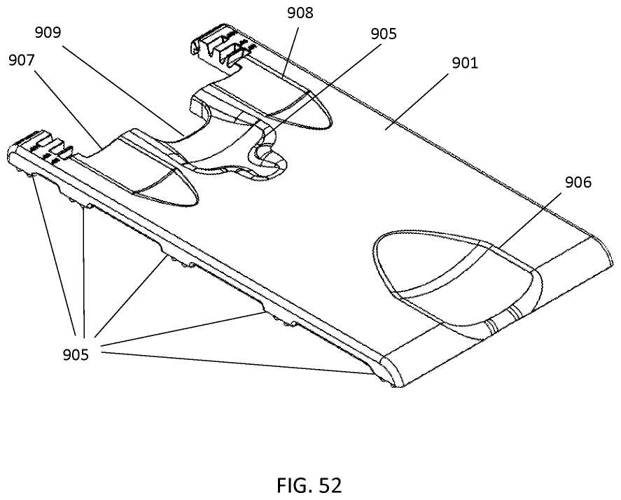

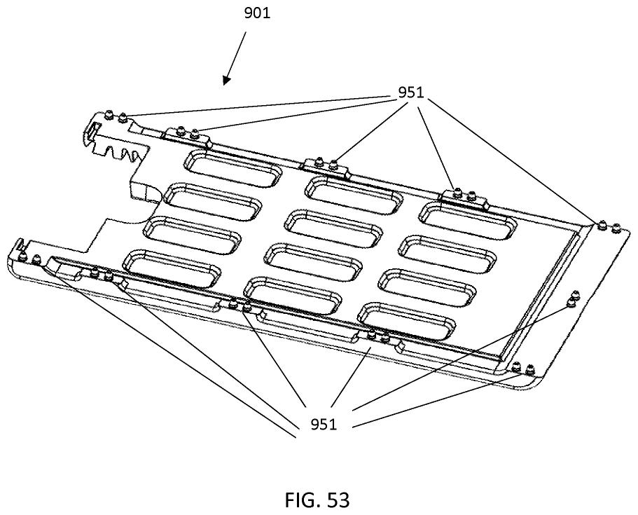

FIG. 52 shows an isometric view from above of calf plate 901. In this view, several pairs of guide pegs 951 are visible. These guide pegs 951 will allow calf plate 901 to be aligned and joined with lower base 902 by inserting into peg receptacles 950. FIG. 53 shows an isometric view of calf plate 901 from the below. All eleven sets of guide pegs 951 are now visible.

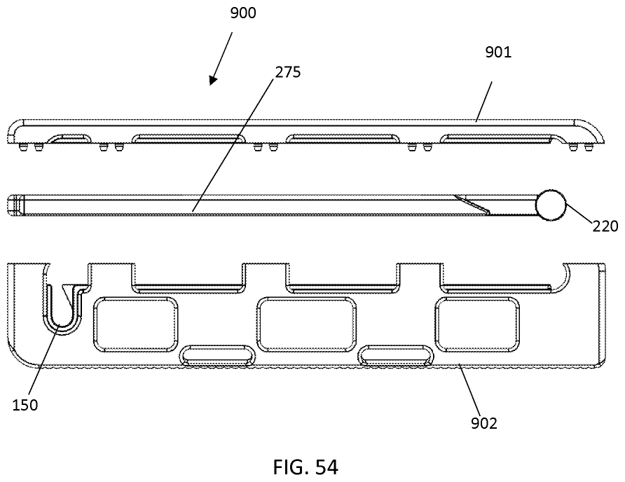

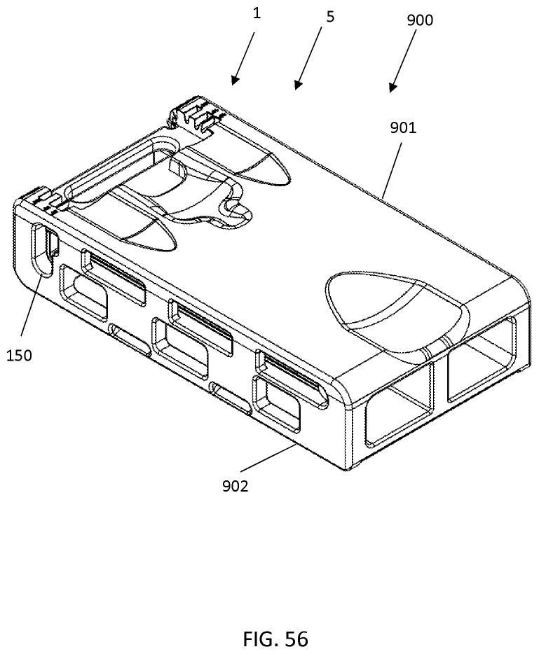

The assembly of base 900 can be visualized in FIG. 54 through FIG. 56. Footpad 275 is placed between calf plate 901 and lower base 902. Lower pin 220 is positioned in a channel created by the lower surface of calf plate 901 and the upper surface of lower base 902. When the guide pegs 951 are inserted into peg receptacles 950, they can be permanently or temporarily joined, allowing for the assembly of base 900. Footpad 275 is now between calf plate 901 and lower base 902, but it can still move from the stowed position 5 of FIG. 56 to the deployed positions shown in other figures.

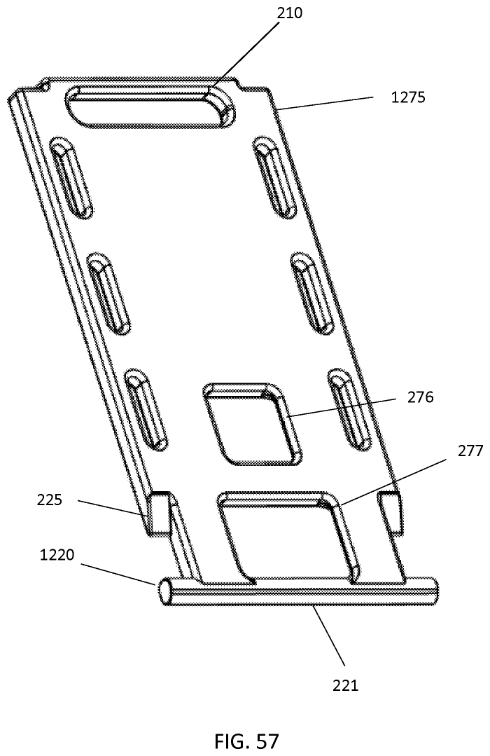

A third embodiment of simulated weightbearing apparatus 1 is depicted in FIG. 57-76. FIG. 57 shows an isometric view of footpad 1275. Footpad 1275 features handle 210 for carrying the footpad 1275 as well as the full simulated weightbearing apparatus when it is fully assembled. Upper access 276 and lower access 277 are through openings in footpad 1275 that allow a physician to work on the plantar surface and hindfoot of a patient. There are two identical positioning edges 225 on either side of footpad 1275, that function the same as previously described. Positioning edge 225 is used to select the angulation of footpad 1275 and to create an opposing force to the load of the patient's foot. Lower pins 1220 have a new design when compared to previous lower pins 220. Lower pins 1220 are non-circular with a major and minor axis and their function will be described later. Shaft 221 is used as a rotation axis for footpad 1275, and in addition connects the lower pins 1220 to each other and to footpad 1275.

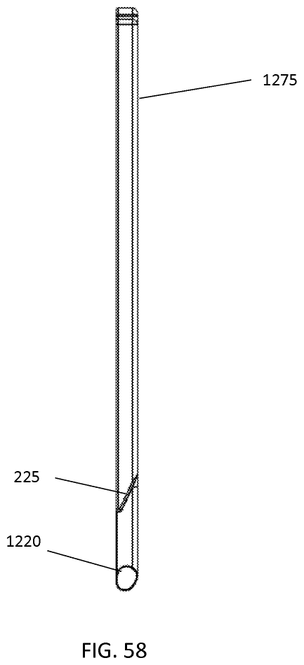

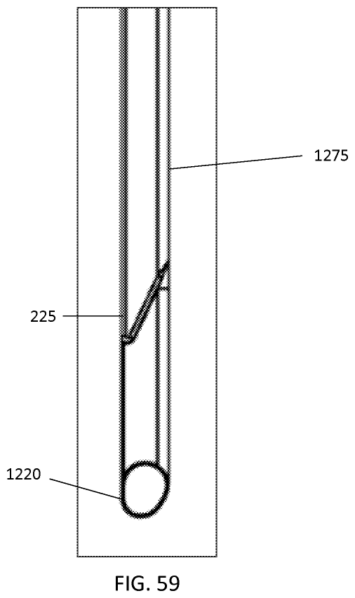

FIGS. 58 and 59 show a side view of footpad 1275 and a close-up view of footpad 1275. The design of lower pin 1220 is now evident. Lower pin 1220 has this design to control the kinematics of the footpad 1275. When footpad 1275 lies horizontally, which is the first rotational orientation, the major axis of lower pin 1220 prevents it from moving downward in the base assembly. When footpad 1275 is then rotated to a second rotational orientation, lower pin 1220 eventually can pass through an opening, which will be described later. Lower pin 1220 may be non-circular, oval, elliptical, keyed, cam-shaped, or otherwise profiled so that the lower pin 1220 can pass through narrow slot 1502 only when footpad 1275 is in a selected rotational orientation.

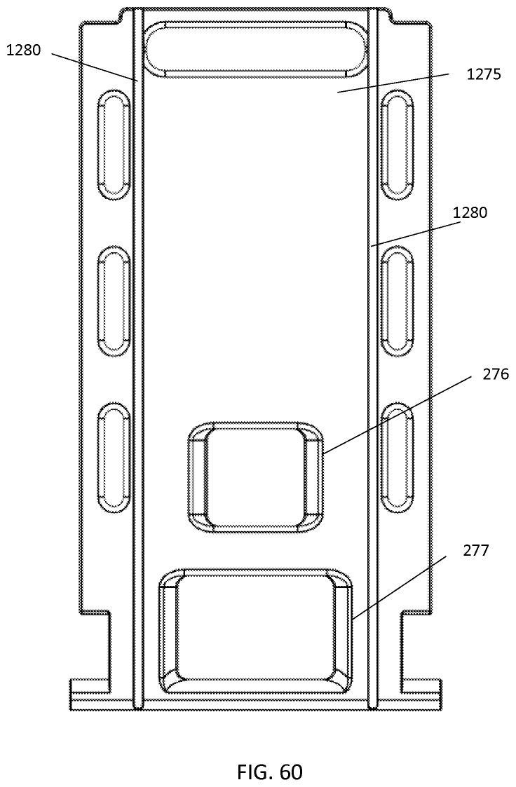

FIG. 60 shows a back view of footpad 1275. This is the opposite side that a patient's foot would contact during use of the simulated weightbearing apparatus. Channels 1280 are visible in this view. Channels 1280 are two identical and symmetrical cylindrical channels in the back of footpad 1275 that guide its motions when it moves horizontally on the base. This will be described in more detail later.

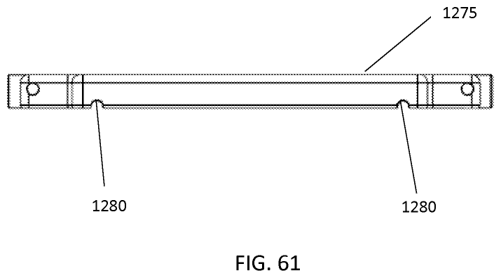

FIG. 61 shows a top view of footpad 1275. Channels 1280 are now easily visible and can be seen as cylindrical channels in footpad 1275.

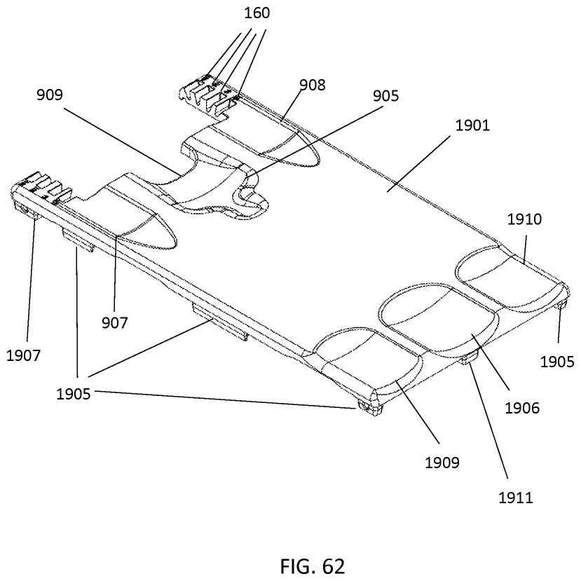

FIG. 62 shows an isometric view of calf plate 1901. Calf plate 1901 has many features that are similar to calf plate 901 but also some new features. There are four sets of locking slots 160, which are symmetrical on either side of calf plate 1901. These four sets of locking slots 160 are designed to receive positioning edges 225 of footpad 1275. Left heel cup 907, heel cup 905, and right heel cup 908 are all contoured depressions in calf plate 1901 designed to receive the heel of a patient. The user can choose to position a foot on the left side, center, or right side of calf plate 1901, such that the hallux, lesser toes, or heel can be positioned in the best spot for the surgeon. There are now corresponding left calf cup 1909, center calf cup 1906, and right calf cup 1910 that allow the patient's entire leg and foot to be moved to one side or the other of calf plate 1901. There are six positioning tabs 1905, three on either side of the bottom of calf plate 1901. These are guidance features to mate calf plate 1901 with lower base 1902. In addition, there are two symmetrical front positioning tabs 1907, that serve as a guidance feature but also as a stop for the motion of footpad 1275. Rear positioning tab 1911 is a guidance feature and a rear stop for the motion of footpad 1275.

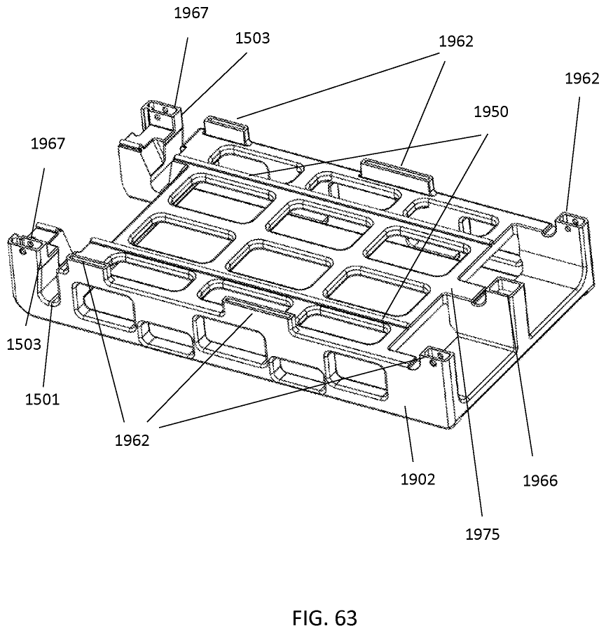

Lower base 1902 is shown in an isometric view in FIG. 63. There is a lattice structure 1975 that forms the main horizontal structure for lower base 1902. There are six positioning receptacles 1962 that receive the positioning tabs 1905 of calf plate 1901, and insure that the two components are properly aligned with each other. Front positioning receptacles 1967 receive front positioning tabs 1907, and rear positioning receptacle 1966 receives rear positioning tab 1911. Footpad stop 1503 is the side wall of front positioning receptacles 1967. Rails 1950 are two rails that run parallel to each other, and mate with channels 1280 of footpad 1275. In this way, footpad 1275 can move horizontally and linearly across lower base 1902, and below calf plate 1901. When footpad 1275 moves linearly, the lower pins 1220 eventually strike footpad stop 1503. The interaction of rails 1950 and channels 1280 are a rail-and-channel guide arrangement that will be described in more detail later. Although rails 1950 are shown on lower base 1902 and channels 1280 are shown on footpad 1275, the arrangement may be reversed, such that a rail is provided on one of the base and the footpad and a channel is provided on the other of the base and the footpad.

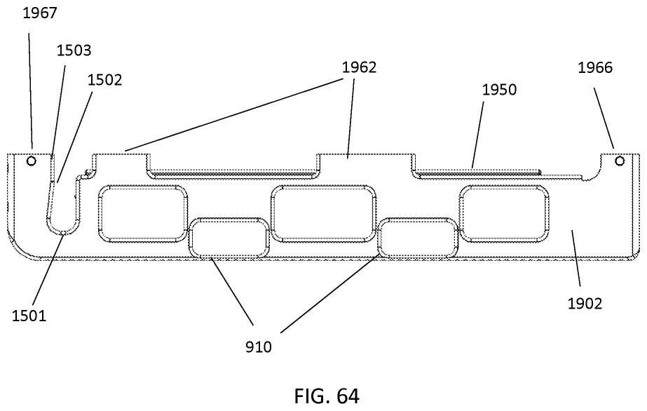

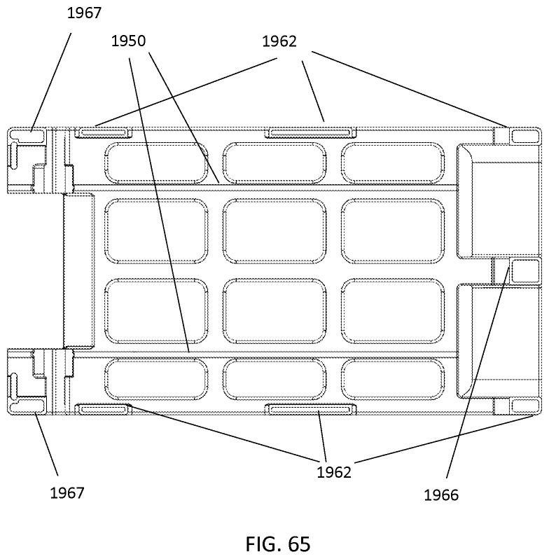

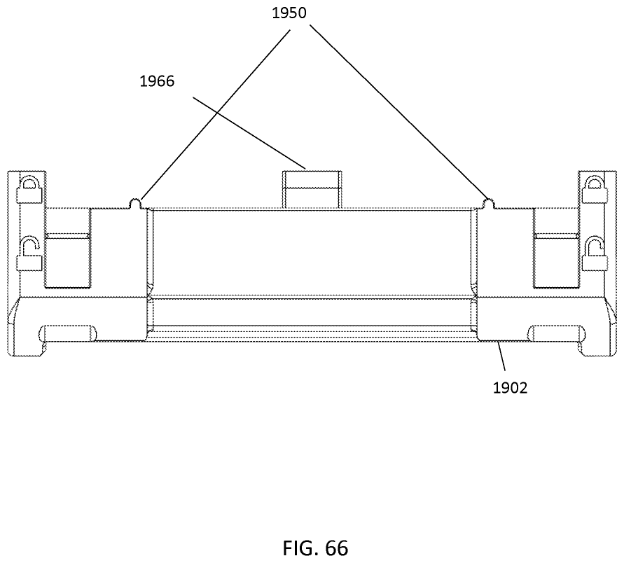

FIG. 64 is a side view of lower base 1902 that shows additional features. Strap slots 910 are visible, which can be used to strap the apparatus to the operating table, or to strap the patient's leg to the apparatus, or both. Narrow slot 1502 is shown at the top of a notch in lower base 1902. Wide slot 1501 is wider than narrow slot 1502 and is an example of a wider receiving region, which is seen near the bottom of lower base 1902. Wide slot 1501 may also be referred to as a wider slot or wider receiving region. The lower pin 1220 of footpad 1275 is designed such that it cannot fit through narrow slot 1502 when footpad 1275 lies horizontally. Only when footpad 1275 is partially rotated upward, can lower pin 1220 eventually slide through narrow slot 1502 such that footpad 1275 can then move downwards. Lower pin 1220 then moves into wide slot 1501 and the wider receiving region where it can anchor and create a counterforce to the load from a patient's foot. FIG. 65 shows a top view of lower base 1902, and FIG. 66 shows an end view of lower base 1902. Rails 1950 are visible. The footpad travels along rails 1950 but also between positioning receptacles 1962. Rear positioning receptacle 1966 extends high enough such that footpad 1275 cannot move past rear positioning receptacle 1966 in that direction.

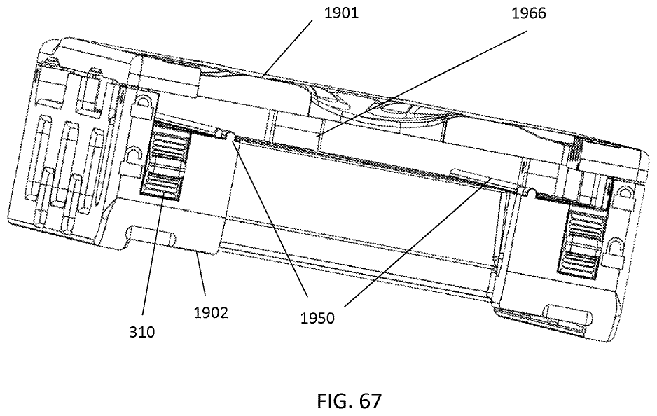

FIG. 67 is an isometric view of lower base 1902 with calf plate 1901, however footpad 1275 is not shown for clarity. Normally footpad 1275 would lie between lower base 1902 and calf plate 1901. In this view, rails 1950 are visible, as well as rear positioning receptacle 1966. Latches 310 assemble to lower base 1902 and serve to retain footpad 1275 when the apparatus is not in use.

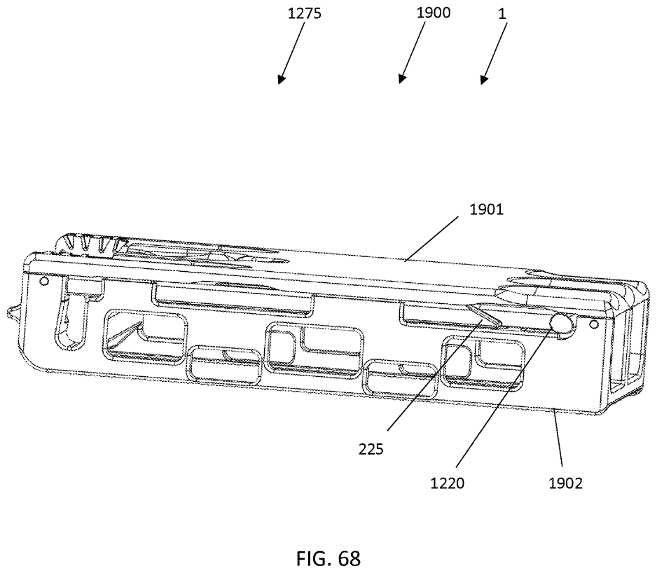

FIG. 68-76 show a sequence of views of the third embodiment of simulated weightbearing apparatus 1 in use. In FIG. 68, the simulated weightbearing apparatus 1 consists of base assembly 1900 (consisting of lower base 1902 and calf plate 1901), footpad 1275, and two latches 310. This is the stowed position of the apparatus, and footpad 1275 is completely stowed between calf plate 1901 and lower base 1902. Lower pin 1220 is visible near one side of lower base 1902. Positioning edge 225 is also visible in the space between lower base 1902 and calf plate 1901. The apparatus can be carried in this position, and sterilized.

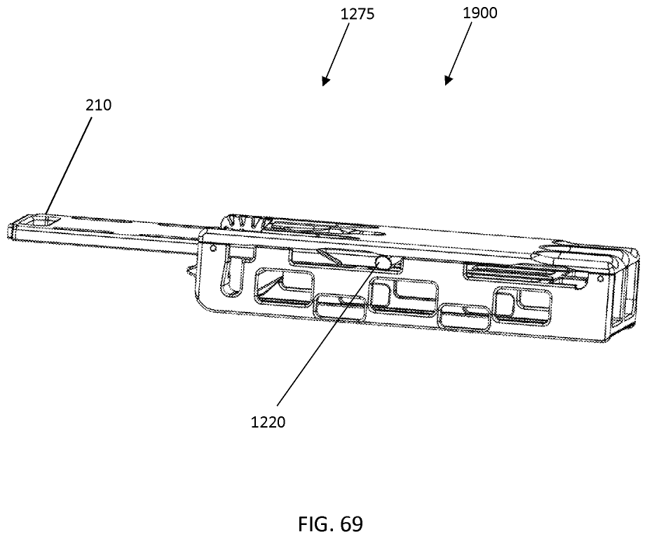

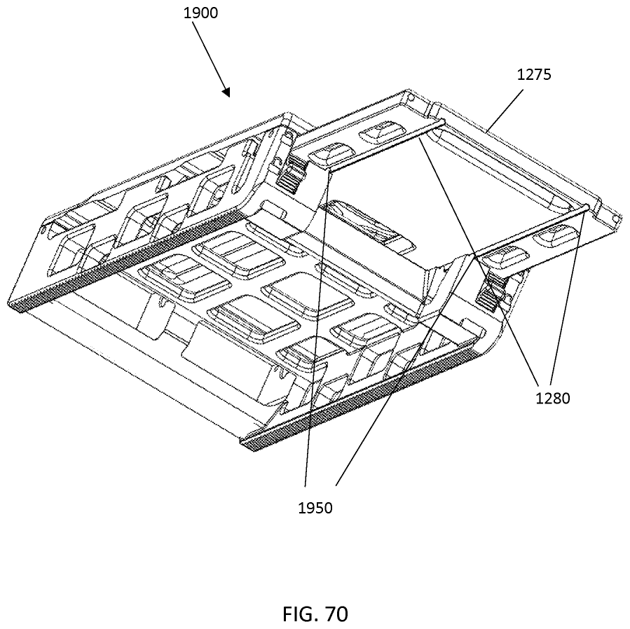

In FIG. 69, the latches 310 have been lowered, releasing footpad 1275. Handle 210 is used to extract footpad 1275 such that it moves horizontally and linearly, with channels 1280 sliding along rails 1950. Footpad 1275 is constrained kinematically to move in a line during this portion of use. FIG. 70 shows an isometric view of the underside of simulated weightbearing apparatus 1, to show channels 1280 sliding along rails 1950. Latches 310 are in the lowered position.

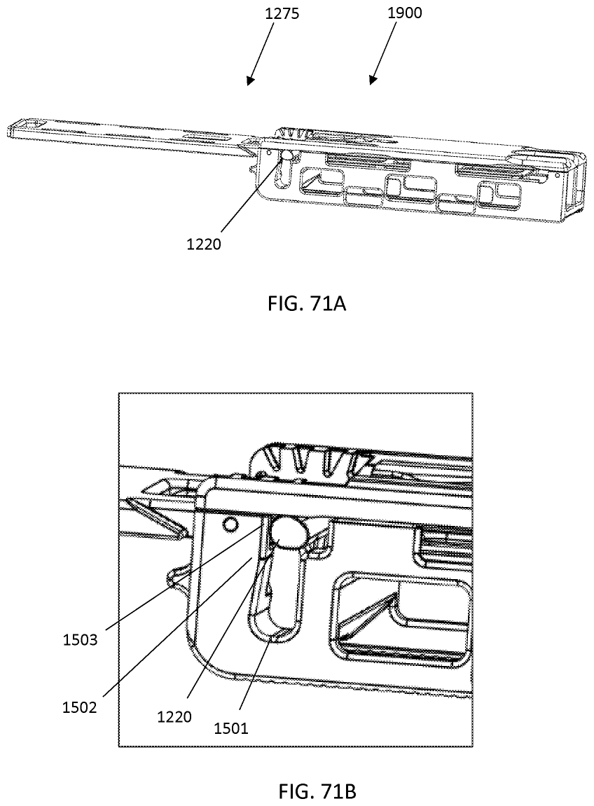

FIG. 71A shows an isometric view and FIG. 71B a close-up view of footpad 1275 as it moves to the end of its linear motion. Now footpad 1275 has struck footpad stops 1503 on both sides, and this is the full extent of its horizontal motion; outward motion is no longer possible. Footpad 1275 cannot move vertically either, because lower pin 1220 and its major axis will not fit through narrow slot 1502 when footpad 1275 lies horizontal. At this time, footpad 1275 can only be either re-stowed into the storage position, or rotated about its shaft 221 and lower pins 1220.

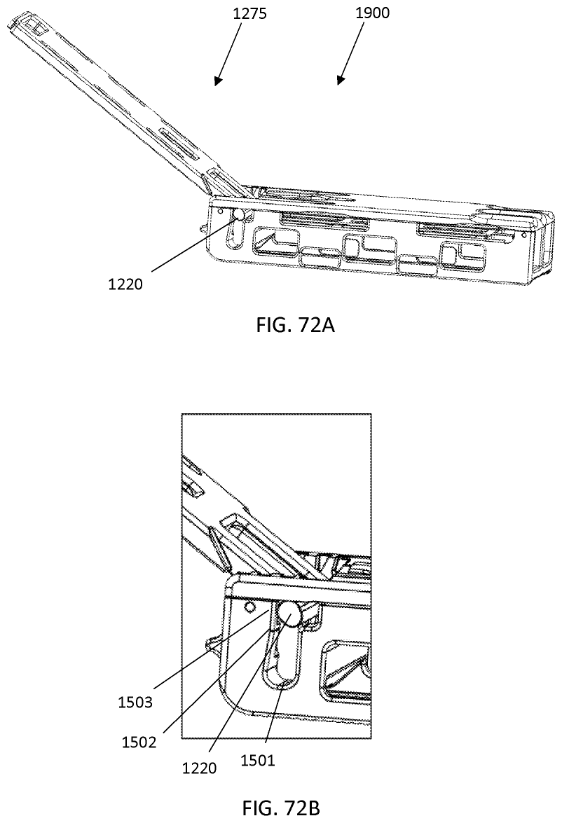

In FIG. 72A and corresponding close-up FIG. 72B, footpad 1275 has now been partially rotated by a user. Lower pins 1220, symmetrical on either side, are now being positioned such that the diameter is smaller than narrow slot 1502. Positioning edge 225 moves around base 1900.

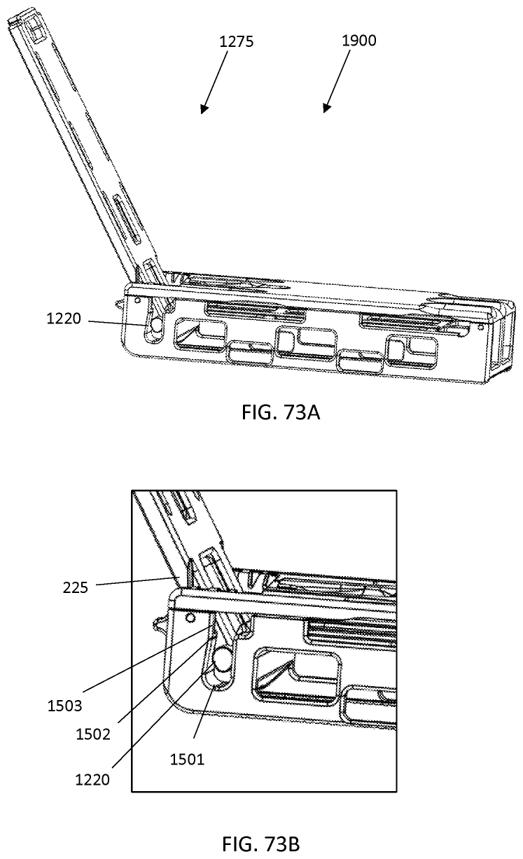

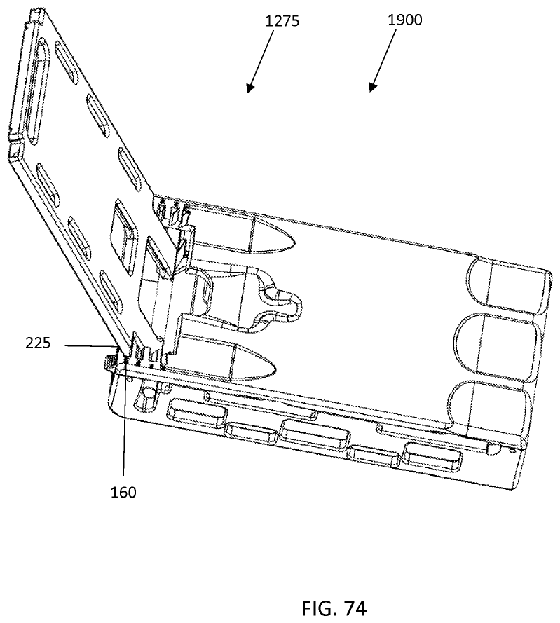

In FIG. 73A and corresponding close-up FIG. 73B, footpad 1275 has now rotated far enough that lower pins 1220 are free to slip through narrow slot 1502 and can move linearly downward. Footpad 1275 and lower pins 1220 move down into wide slot 1501. Positioning edges 225 move towards one of the pairs of locking slots 160. In FIG. 73B, positioning edges 225 have been inserted into the most angled position of locking slot 160. Kinematic motion is now stopped for footpad 1275. A resistive couple moment can be created between the force created at positioning edge 225 and locking slot 160, on the top, and lower pins 1220 and wide slot 1501, on the bottom. FIG. 74 shows an isometric view of the same position as FIGS. 73A and 73B, showing the engagement of positioning edge 225 and locking slot 160.

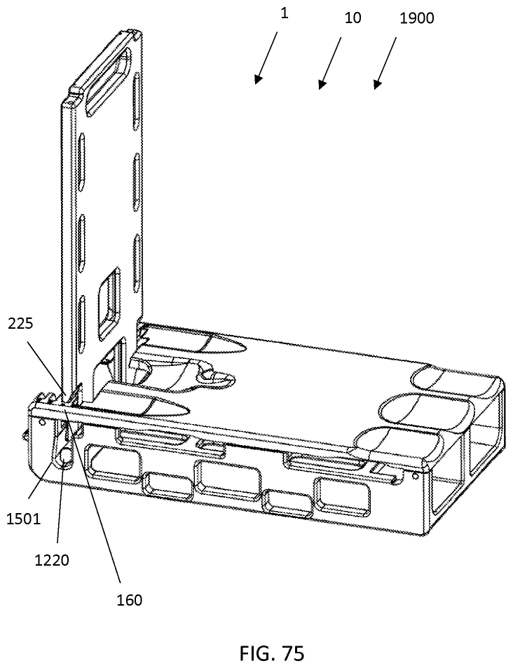

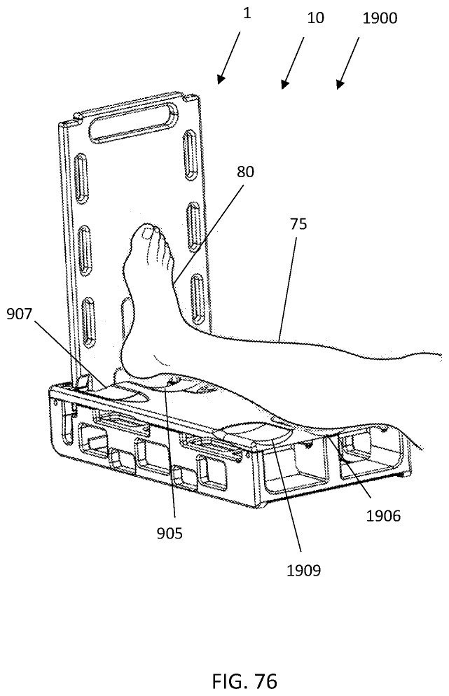

FIGS. 75 and 76 show an isometric view of simulated weightbearing apparatus 1 in upright position 10. In this instance, the footpad 1275 has been rotated further before positioning edges 225 engage locking slot 160, in the perpendicular position. In FIG. 76, a representative patient's leg 75 with foot 80 has been positioned in the center position for surgery or imaging. It could be moved to the left or to the right in order to accomplish the aims of the physician. The foot and leg of the patient can be moved during the procedure if the best location changes during the procedure. In addition, the footpad 1275 can be moved to various angles, from forward leaning to rearward reclining, throughout the procedure to best accomplish the aims of the physician. The footpad 1275 can also be stowed repeatedly during the procedure when it is not needed.

The simulated weightbearing apparatus 1 has controlled kinematics to be most useful to the physician. Initially, the footpad 1275 is constrained to linear horizontal motion until it reaches the stop. Then, to continue usage, it requires rotation to a desired angle. Finally, the lower part of the footpad 1275 then moves downward into a supporting position at the desired angle. If the simulated weightbearing apparatus 1 is placed on one or more wedges 1000, then the kinematics can change slightly. For example, the initial footpad 1275 motion of linear horizontal motion may be angled linear motion if a wedge 1000 is underneath apparatus 1. But the same constraints described above still apply throughout the motion.

The simulated weightbearing apparatus 1 has the following features.

### Features of the System

Fully radiolucent except marker strips on footpad to simulate ground.
Reproducible imaging with a flat surface pressing on the foot.
Quick set and release of the footpad.
Multi-use (autoclavable for operating room use, or non-sterile use).
Operates in different clinical settings: intraoperative, post-op, clinic.
Folds flat to a working surface.
Portable, with a handle.
Allows entire foot visualization.
Patient comfort features such as rounded edges, cushions, etc.
Footpad able to adjust to various angles.
Can be used for A/P or lateral imaging with no reassembly.
Footpad features include heel support, guiderails for foot, texture, and holes for cleaning.
Slots to receive belts for the table, leg, or foot, to stabilize the system.
Slots in the footpad to allow mounting of surgical instruments.
A separate kit of instruments that can be used with the apparatus.
A latch that slides up and down to hold the footpad in a stowed position.
One or more wedges that can be used to create an incline or elevate the apparatus.
Controlled kinematics of the footpad for smooth operation, including any of the following: linear extraction, constrained rotation, downward movement of the lower pin into a locking slot, and creation of a resistive couple between the lower pin and positioning edge.
Adjustable foot orientation in numerous angles of the footpad.
Radiolucent and reproducible imaging.
Hands-free maintenance of a selected simulated weight-bearing orientation and, in some embodiments, a selected loading condition.
Wedge-based elevation and/or inclination.
Attachment points for surgical guides and instruments.
Surgical corrections or imaging can be at different foot angulations, or with different amounts of foot loading. The foot can be in different locations, left, center or right, on the apparatus.
The access openings, slots, radiolucent materials, and instrument mounting features may permit imaging and surgical access while the patient's foot is maintained in the simulated weight-bearing position.

A method of use for using the simulated weight-bearing apparatus in surgery is described here. A patient is prepped for surgery according to physician's instructions. A sterile field is established. The simulated weight-bearing apparatus and any wedges can be sterilized, if desired. One or more wedges 1000 are placed at the end of the bed, and can be used to either create an angled surface or elevated surface for the simulated weightbearing apparatus. Sterile apparatus 1 in stowed position is placed on the bed or on the wedges, as desired. Patient's leg is placed on top of the stowed apparatus, with footpad 275 or 1275 in stowed position 5. When desired, the footpad 275 or 1275 is deployed through the controlled kinematics described above to any deployed position. Imaging studies or surgery are performed. Instruments can be attached to footpad 200, 275, or 1275 and used, and then removed when desired. The footpad is then collapsed and re-stowed into the stowed position. This cycle of deploying and re-stowing the apparatus can be repeated any number of times during a surgical procedure.

Optionally, footpad 250 can be used. Orient the upper footpad 252 and lower footpad 251 to achieve the desired positioning or loading on the patient's foot. By moving upper footpad 252 relative to lower footpad 251, different regions of the patient's foot may be positioned, oriented, or loaded differently.

The locking and positioning features described may be implemented in numerous ways. In the embodiments, the footpad is stabilized in a deployed position by interaction between an upper engagement feature and a lower engagement feature. The upper engagement feature may include positioning edge 225, and the lower engagement feature may include lower pin 220 or lower pin 1220. When the footpad is deployed, positioning edges 225 engage one pair of the locking slots 160, while lower pin 220 or 1220 engages corner slot 150 or wide slot 1501. In this configuration, the force applied by the patient's foot to the footpad is resisted by spaced-apart reaction forces at the upper and lower engagement features. The spaced-apart reaction forces create a resistive couple that stabilizes the footpad relative to the base.

In some embodiments, the locking mechanism does not require a separate latch, clamp, fastener, screw, or manually tightened component to hold the footpad in the deployed position. Instead, the geometry of the footpad and base creates a self-supporting deployed configuration. The load applied to the footpad by the patient's foot may urge the positioning edge more securely into the selected locking slot and may urge the lower pin more securely into the corner slot or wide slot. Thus, the simulated weightbearing apparatus can maintain the selected footpad angle in a hands-free manner after the footpad has been deployed.

The locking slots 160 may be arranged at different locations along the base to provide different angular positions of the footpad. Selection of a particular locking slot 160 determines the angular orientation of the footpad relative to the calf plate. The selected angular orientation may also be referred to as a selected angle or selected deployed angle of the footpad relative to the base. The locking slots 160 may be positioned to provide a perpendicular orientation, a forward-leaning orientation, a rearward-leaning orientation, or any other desired orientation. The locking slots 160 may be provided in pairs on opposite sides of the base so that the positioning edges 225 on both sides of the footpad engage corresponding locking slots 160 simultaneously.

The positioning edge 225 may be any structure capable of engaging the base to resist rotation or translation of the footpad. For example, positioning edge 225 may be a rib, tab, flange, projection, shoulder, boss, pin, rail, protrusion, recess, detent, tooth, hook, or other engagement feature. Similarly, locking slot 160 may be any complementary engagement structure, including a slot, groove, notch, pocket, recess, detent, channel, aperture, shoulder, or other receiving feature.

The positioning edge and locking slot may be reversed between the footpad and the base, such that the footpad includes a receiving feature and the base includes a projection or other engagement feature.

The lower pin 220 or 1220 may also be any structure capable of guiding, supporting, or locking the lower portion of the footpad. For example, the lower pin may be a cylindrical pin, non-circular pin, oval pin, elliptical pin, keyed pin, cam follower, roller, wheel, axle, boss, projection, rib, rail, tab, or other follower. The corresponding structure in the base may be a receiving region, base slot, corner slot, wide slot, narrow slot, guide channel, track, groove, recess, notch, cam surface, or other guide feature. The lower pin and corresponding guide feature may cooperate to guide movement of the footpad during deployment and to stabilize the footpad after deployment. The corresponding structure in the base may also be referred to as a corresponding receiving feature configured to receive, guide, support, or retain the lower engagement feature.

In some embodiments, the lower pin 1220 is shaped so that it permits movement of the footpad only after the footpad has reached a selected rotational orientation. For example, lower pin 1220 may have a non-circular profile that prevents the lower pin from passing through narrow slot 1502 when the footpad is substantially horizontal. After the footpad is rotated, a smaller dimension of the lower pin 1220 may align with narrow slot 1502, allowing the lower pin 1220 to pass through narrow slot 1502 and move into wide slot 1501. In this manner, the lower pin 1220 and narrow slot 1502 cooperate as a keyed or cam-like mechanism that controls the sequence of footpad motion.

The controlled motion of the footpad may include multiple stages. In a first stage, a first guide feature guides a first movement of the footpad, such as generally linear movement or linear extraction relative to the base while the footpad remains in a stowed or partially stowed orientation. In a second stage, a second guide feature permits or guides rotation of the footpad relative to the base after the footpad reaches a stop or end of travel. For example, the second guide feature may include lower pin 1220, shaft 221, narrow slot 1502, wide slot 1501, a clearance space or pivot region, a cam surface, or another structure that permits or guides rotation of the footpad after the first movement. In a third stage, a lower portion of the footpad moves downward into a locking or supporting region of the base. In a fourth stage, positioning edge 225 engages one of the locking slots 160 to select the desired angular orientation. This sequence allows the footpad to be stored compactly within the base, deployed predictably, and locked at a selected deployed angle and position without requiring complex assembly by the user.

The guide and locking features may be configured so that the footpad cannot move directly from the stowed position to the locked deployed position without first undergoing a predetermined sequence of translation and rotation. This controlled kinematic sequence may reduce unintended deployment, improve ease of use, prevent misalignment, and provide repeatable positioning of the footpad. The same guide and locking features may also permit the footpad to be repeatedly deployed and re-stowed during a procedure without removing the apparatus from the patient or from the operating table.

The locking mechanism may include one or more stops to limit motion of the footpad. For example, footpad stop 1503 may limit outward linear movement of footpad 1275. Rear positioning receptacle 1966 or rear positioning tab 1911 may limit rearward motion of the footpad. Narrow slot 1502 may limit downward motion until lower pin 1220 is rotationally aligned with narrow slot 1502. Wide slot 1501 may receive lower pin 1220 after the footpad has rotated sufficiently. These structures may cooperate to define a repeatable deployment path.

The guide and locking structures or locking features may be symmetrical on the left and right sides of the apparatus. The locking feature may include positioning edge 225, locking slot 160, lower pin 220/1220, corner slot 150, narrow slot 1502, wide slot 1501, or combinations thereof. Symmetrical engagement of positioning edges 225 with locking slots 160, and symmetrical engagement of lower pins 220 or 1220 with corresponding slots, may resist twisting of the footpad relative to the base. In other embodiments, the guide and locking structures may be asymmetric, offset, or provided on only one side of the apparatus, depending on the desired loading, access, imaging, or manufacturing requirements.

The locking mechanism may be designed to resist forces applied by the patient's foot during simulated weightbearing, including forces generally perpendicular to the footpad, forces tending to rotate the footpad, and forces tending to translate the footpad relative to the base. The engagement between the positioning edge and locking slot may resist movement of the upper portion of the footpad, while the engagement between the lower pin and corresponding lower slot may resist movement of the lower portion of the footpad. Together, these spaced-apart engagements maintain the footpad in the selected orientation.

In some embodiments, the locking mechanism may be releasable by reversing the deployment sequence. For example, the user may lift or rotate the footpad to disengage positioning edge 225 from locking slot 160, move lower pin 1220 out of wide slot 1501 and through narrow slot 1502, rotate the footpad toward the stowed orientation, and then slide the footpad linearly back into the base. The footpad may then be retained in the stowed position by latches 310 or by another retention mechanism.

Although the present invention has been described in terms of the foregoing preferred embodiment, such description has been for exemplary purposes only and, as will be apparent to those of ordinary skill in the art, many alternatives, equivalents, and variations of varying degrees will fall within the scope of the present invention. That scope, accordingly, is not to be limited in any respect by the foregoing detailed description; rather, it is defined only by the claims that follow.

What is claimed is:

1. A simulated weight-bearing positioning apparatus for positioning a patient's foot, comprising:
a base configured to support at least a portion of a patient's leg;
a footpad movable relative to the base between a stowed position and a deployed position, the footpad having a patient-contacting surface configured to contact a plantar surface of the patient's foot;
a first guide feature configured to guide the footpad through a first movement relative to the base;
a second guide feature configured to permit the footpad to rotate relative to the base after the first movement;
a locking feature configured to retain the footpad in the deployed position at a selected angle relative to the base; and
wherein movement of the footpad from the stowed position to the deployed position includes at least a translation of the footpad relative to the base followed by rotation of the footpad relative to the base.

2. The apparatus of claim 1, wherein the first guide feature comprises a rail on one of the base and the footpad and a channel on the other of the base and the footpad.

3. The apparatus of claim 1, wherein the base comprises a calf plate and a lower base, and wherein the footpad is at least partially received between the calf plate and the lower base in the stowed position.

4. The apparatus of claim 1, wherein the locking feature comprises a positioning edge on the footpad and a locking slot on the base.

5. The apparatus of claim 4, wherein the base includes a plurality of locking slots corresponding to a plurality of angular positions of the footpad, including at least one forward-leaning position, an upright position, and at least one rearward-leaning position.

6. The apparatus of claim 1, further comprising a lower engagement feature on the footpad configured to move into a corresponding receiving feature of the base when the footpad is in the deployed position, wherein the lower engagement feature comprises a lower pin and the corresponding receiving feature comprises a slot.

7. The apparatus of claim 1, wherein the base includes a stop configured to limit translation of the footpad before the footpad rotates relative to the base.

8. The apparatus of claim 1, wherein the locking feature stabilizes the footpad using spaced-apart reaction forces at an upper engagement feature and a lower engagement feature when a force is applied by the patient's foot to the footpad.

9. The apparatus of claim 1, further comprising at least one wedge configured to support the apparatus in an inclined orientation or an elevated orientation.

10. The apparatus of claim 1, further comprising at least one instrument mounting feature coupled to the footpad and configured to support or guide a surgical instrument while the patient's foot is held in the simulated weight-bearing position.

11. The apparatus of claim 1, wherein the footpad comprises an upper footpad portion, a lower footpad portion, and a pivot coupling the upper footpad portion to the lower footpad portion so that different regions of the patient's foot may be positioned, oriented, or loaded differently.

12. A simulated weight-bearing positioning apparatus, comprising:
a base comprising a narrow slot and a wider receiving region;
a footpad movable relative to the base between a stowed position and a deployed position;
a non-circular lower pin coupled to the footpad; and
wherein the non-circular lower pin is configured to prevent downward movement of the footpad through the narrow slot when the footpad is in a first rotational orientation, and to permit downward movement of the footpad through the narrow slot when the footpad is in a second rotational orientation.

13. The apparatus of claim 12, wherein the non-circular lower pin has a major axis and a minor axis.

14. The apparatus of claim 13, wherein the major axis prevents the non-circular lower pin from passing through the narrow slot when the footpad is substantially horizontal, and wherein the minor axis permits the non-circular lower pin to pass through the narrow slot after the footpad has been rotated.

15. The apparatus of claim 12, wherein the wider receiving region is configured to receive the non-circular lower pin after the non-circular lower pin passes through the narrow slot, and wherein a positioning edge on the footpad is configured to engage a locking slot of the base after the non-circular lower pin moves into the wider receiving region.

16. The apparatus of claim 12, wherein the non-circular lower pin and the narrow slot cooperate to require a predetermined sequence of translation, rotation, and downward movement before the footpad reaches the deployed position.

17. A method of positioning a patient's foot in a simulated weight-bearing position, comprising:
placing a simulated weight-bearing apparatus beneath at least a portion of a patient's leg, the simulated weight-bearing apparatus comprising a base and a footpad movable relative to the base;
moving the footpad from a stowed position relative to the base through a first movement;
rotating the footpad relative to the base after the first movement;
engaging the footpad with the base in a deployed position at a selected angle;
positioning a plantar surface of the patient's foot against the footpad; and
maintaining the patient's foot in a simulated weight-bearing position while imaging or surgery is performed.

18. The method of claim 17, further comprising moving a lower pin of the footpad downward into a receiving region of the base after rotating the footpad, and engaging a positioning edge of the footpad with one of a plurality of locking slots of the base.

19. The method of claim 17, further comprising re-stowing the footpad during the surgical procedure and subsequently redeploying the footpad during the same surgical procedure without removing the base from beneath the patient's leg.

20. The method of claim 17, further comprising at least one of attaching a surgical instrument guide to the footpad, placing one or more wedges beneath the simulated weight-bearing apparatus, or using a radiopaque marker strip on or within the footpad as a reference during imaging.
